(19) United States
(12) Patent Application Publication
(10) Pub. No.: US 2026/0232766 A1
(43) Pub. Date: Aug. 13, 2026

(54) METHODS AND COMPOSITIONS FOR COMBINATION THERAPY

(71) Applicants: SIMCHA IL-18, INC., NEW HAVEN, CT (US); MSD INTERNATIONAL BUSINESS GMBH, LUCERNE (CH)

(72) Inventors: AARON RING, NEW HAVEN, CT (US); BEATRICE MCQUEEN, NEW HAVEN, CT (US); JEREMY BARTON, NEW HAVEN, CT (US); JEFFREY N. LINDQUIST, NEW HAVEN, CT (US); HIRDESH UPPAL, NEW HAVEN, CT (US)

(73) Assignees: SIMCHA IL-18, INC., NEW HAVEN, CT (US); MSD INTERNATIONAL BUSINESS GMBH, LUCERNE (CH)

(21) Appl. No.: 19/149,391

(22) PCT Filed: Jan. 23, 2024
(86) PCT No.: PCT/US2024/012655

§ 371 Date: Jul. 18, 2025

Related U.S. Application Data
(60) Provisional application No. 63/481,214, filed on Jan. 24, 2023.
(60) Provisional application No. 63/488,384, filed on Mar. 3, 2023.
(60) Provisional application No. 63/488,473, filed on Mar. 3, 2023.
(60) Provisional application No. 63/511,404, filed on Jun. 30, 2023.

(51) Int. Cl. A61K 38/20; A61K 31/138; A61K 31/167; A61K 31/192; A61K 31/405; A61K 31/426; A61K 39/00; A61P 35/00; A61P 35/02; A61P 37/02; C07K 16/28

(52) U.S. Cl. CPC A61K 38/20; A61K 31/138; A61K 31/167; A61K 31/192; A61K 31/405; A61K 31/426; A61P 35/00; A61P 35/02; A61P 37/02; C07K 16/2818; A61K 2039/505; A61K 2039/545; C07K 2317/565; C07K 2317/76

(57) ABSTRACT

The present disclosure provides compositions and methods for treating a disease, such as cancer or a tumor, comprising: administering successive doses of an anti-PD-1 antibody composition comprising an anti-PD-1 antibody, or antigen binding fragment thereof, such as pembrolizumab or a pembrolizumab variant to the subject and administering successive doses of a decoy-resistant (DR) IL-18 composition comprising a DR IL-18 polypeptide to the subject; thereby improving the condition of the subject, such as e.g., causing immunotherapy-induced regression of the disease in the subject, such as immunotherapy-induced regression of a cancer or tumor.

### CROSS-REFERENCE

This application claims benefit of U.S. Provisional Patent Application Nos. 63/481,214 filed Jan. 24, 2023, 63/488,384 filed Mar. 3, 3023, 63/488,473 filed Mar. 3, 2023, and 63/511,404 filed Jun. 30, 2023, each of which applications are incorporated herein by reference in their entirety.

### REFERENCE TO SEQUENCE LISTING SUBMITTED ELECTRONICALLY

This instant application contains a Sequence Listing which has been submitted electronically in XML format and is hereby incorporated by reference in its entirety. Said XML copy, created Jan. 12, 2024, is named “ST-011-WO1_seq_list.xml” and is 35 kb in size.

### BACKGROUND

PD-1 is recognized as an important player in immune regulation and the maintenance of peripheral tolerance. PD-1 is moderately expressed on naive T, B and NKT cells and up-regulated by T/B cell receptor signaling on lymphocytes, monocytes and myeloid cells (Sharpe et al., The function of programmed cell death 1 and its ligands in regulating autoimmunity and infection. Nature Immunology, 8:239-245 (2007)).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong et al., Nat Med. 8 (8): 793-800 (2002); Yang et al. Invest Ophthalmol Vis Sci. 49:2518-2525 (2008); Ghebeh et al. Neoplasia 8:190-198 (2006); Hamanishi et al., Proc. Natl. Acad. Sci. USA 104:3360-3365 (2007); Thompson et al., Cancer 5:206-211 (2006); Nomi et al., Clin. Cancer Research 13:2151-2157 (2007); Ohigashi et al., Clin. Cancer Research 11:2947-2953 (2005); Inman et al., Cancer 109:1499-1505 (2007); Shimauchi et al. Int. J. Cancer 121:2585-2590 (2007); Gao et al. Clin. Cancer Research 15:971-979 (2009); Nakanishi J. Cancer Immunol Immunother. 56:1173-1182 (2007); and Hino et al., Cancer 00:1-9 (2010)).

Similarly, PD-1 expression on tumor infiltrating lymphocytes was found to mark dysfunctional T cells in breast cancer and melanoma (Ghebeh et al., BMC Cancer. 8:5714-15 (2008); Ahmadzadeh et al., Blood 114:1537-1544 (2009)) and to correlate with poor prognosis in renal cancer (Thompson et al., Clinical Cancer Research 15:1757-1761 (2007)). Thus, it has been proposed that PD-L1 expressing tumor cells interact with PD-1 expressing T cells to attenuate T cell activation and evasion of immune surveillance, thereby contributing to an impaired immune response against the tumor.

Immune checkpoint therapies targeting the PD-1 axis have resulted in groundbreaking improvements in clinical response in multiple human cancers (Brahmer et al., N Engl J Med 2012, 366:2455-65; Garon et al. N Engl J Med 2015, 372:2018-28; Hamid et al., N Engl J Med 2013, 369:134-44; Robert et al., Lancet 2014, 384:1109-17; Robert et al., N Engl J Med 2015, 372:2521-32; Robert et al., N Engl J Med 2015, 372:320-30; Topalian et al., N Engl J Med 2012, 366:2443-54; Topalian et al., J Clin Oncol 2014, 32:1020-30; Wolchok et al., N Engl J Med 2013, 369:122-33). Immune therapies targeting the PD-1 axis include monoclonal antibodies directed to the PD-1 receptor KEYTRUDA® (pembrolizumab), Merck Sharp & Dohme LLC., Rahway, NJ, USA; OPDIVO® (nivolumab), Bristol-Myers Squibb Company, Princeton, NJ, USA, and LIBTAYO® (cemiplimab), Regeneron Pharmaceuticals, Inc., Tarrytown, NY, USA) and also those that bind to the PD-L1 ligand (MPDL3280A; TECENTRIQ® (atezolizumab), Genentech, San Francisco, CA, USA; IMFINZI® (durvalumab), AstraZeneca Pharmaceuticals LP, Wilmington, DE; BAVENCIO® (avelumab), Merck KGaA, Darmstadt, Germany; JEMPERLI® (dostarlimab), GlaxoSmithKline Biologics LLC, Philadelphia, PA, USA. Both therapeutic approaches have demonstrated anti-tumor effects in numerous cancer types.

Interleukin-18 (IL-18) is a pro-inflammatory cytokine that can stimulate T cells, NK cells, and myeloid cells. IL-18 has been proposed as an immunotherapeutic agent for the treatment of cancer, given its ability to stimulate anti-tumor cells. However, the clinical efficacy of IL-18 has been limited.

Thus, there is a need for compositions and methods that provide effective IL-18 signaling activity to treat and prevent cancer and other diseases and disorders.

### SUMMARY

One aspect of the present disclosure provides a method of treating a disease in a subject in need thereof comprising: (a) administering successive doses of an anti-PD-1 antibody composition comprising pembrolizumab to the subject; and (b) administering successive doses of a decoy-resistant (DR) IL-18 composition comprising a polypeptide to the subject, wherein the polypeptide is a modified IL-18 polypeptide comprising an amino acid sequence set forth in any one of SEQ ID NOs: 36-43; thereby causing immunotherapy-induced regression of the disease in the subject.

One aspect of the present disclosure provides a method of treating a disease in a subject in need thereof comprising: (a) administering successive doses of an anti-PD-1 antibody composition comprising pembrolizumab to the subject; and (b) administering successive doses of a decoy-resistant (DR) IL-18 composition comprising a polypeptide comprising an amino acid sequence set forth in SEQ ID NO: 41 to the subject.

In some embodiments, the method further comprises administering cytokine release syndrome (CRS) prophylaxis to the subject with or before a dose of the successive doses of the DR IL-18 composition. In some embodiments, the method further comprises administering a dose of a CRS prophylactic agent to the subject with or before a dose of the successive doses of the DR IL-18 composition. In some embodiments, the method comprises administering CRS prophylaxis to the subject with or before each dose of the successive doses of the DR IL-18 composition. In some embodiments, the method comprises administering a dose of a CRS prophylactic agent to the subject with or before each dose of the successive doses of the DR IL-18 composition. In some embodiments, the dose of the CRS prophylaxis comprises at least one of a NSAID, acetaminophen, diphenhydramine, histamine H1 antagonist, famotidine, H2 blocker, or fluids administered to the subject. In some embodiments, the CRS prophylaxis comprises administering to the subject at least a NSAID and acetaminophen; at least a NSAID and a H1 antagonist; at least acetaminophen and a H1 antagonist; or at least a NSAID, acetaminophen, and a H1 antagonist. In some embodiments, the dose of the CRS prophylaxis is administered to the subject orally or intravenously.

In some embodiments, each dose of the successive doses of the anti-PD-1 antibody composition is administered to the subject Q3W. In some embodiments, each dose of the successive doses of the anti-PD-1 antibody composition is administered to the subject Q3W. In some embodiments, each dose of the successive doses of the anti-PD-1 antibody composition is administered to the subject about every 21 days. In some embodiments, each dose of the successive doses of the anti-PD-1 antibody composition comprises a fixed dose of 200 mg Pembrolizumab. In some embodiments, each dose of the successive doses of the anti-PD-1 antibody composition comprises about 2 mg/kg pembrolizumab per body weight of the subject. In some embodiments, each dose of the successive doses of the anti-PD-1 antibody composition is administered to the subject Q3W as a fixed dose of 200 mg pembrolizumab. In some embodiments, each dose of the successive doses of the anti-PD-1 antibody composition is administered to the subject about every 21 days as a fixed dose of 200 mg pembrolizumab. In some embodiments, each dose of the successive doses of the anti-PD-1 antibody composition is administered to the subject Q3W as a dose of about 2 mg/kg pembrolizumab per body weight of the subject. In some embodiments, each dose of the successive doses of the anti-PD-1 antibody composition is administered to the subject about every 21 days a dose of about 2 mg/kg pembrolizumab per body weight of the subject. In some embodiments, each dose of the successive doses of the DR IL-18 composition is administered to the subject weekly. In some embodiments, each dose of the successive doses of the DR IL-18 composition is administered to the subject about every 7 days. In some embodiments, a next dose of the DR IL-18 composition is administered to the subject at least 6 days after a previous dose of the DR IL-18 composition. In some embodiments, a next dose of the DR IL-18 composition is administered to the subject at most 9 days after a previous dose of the DR IL-18 composition. In some embodiments, the polypeptide is at a concentration of about 30 mg/mL. In some embodiments, a dose of the DR IL-18 composition comprises at least about 15 μg/kg of the polypeptide per body weight of the subject. In some embodiments, a dose of the DR IL-18 composition comprises at least about 20 μg/kg of the polypeptide per body weight of the subject. In some embodiments, a dose of the DR IL-18 composition comprises at least about 30 μg/kg of the polypeptide per body weight of the subject. In some embodiments, a dose of the DR IL-18 composition comprises at least about 90 μg/kg of the polypeptide per body weight of the subject. In some embodiments, a dose of the DR IL-18 composition comprises at least about 180 μg/kg of the polypeptide per body weight of the subject. In some embodiments, a dose of the DR IL-18 composition comprises at least about 360 μg/kg of the polypeptide per body weight of the subject. In some embodiments, a dose of the DR IL-18 composition comprises at least about 600 μg/kg of the polypeptide per body weight of the subject. In some embodiments, a dose of the DR IL-18 composition comprises at least about 900 μg/kg of the polypeptide per body weight of the subject. In some embodiments, a dose of the DR IL-18 composition comprises at least about 1200 μg/kg of the polypeptide per body weight of the subject. In some embodiments, the dose of the DR IL-18 composition is at least one of an initial dose, a first dose, or a lowest dose administered to the subject. In some embodiments, each dose of the DR IL-18 composition comprises between about 15 μg/kg to about 1200 μg/kg of the polypeptide per body weight of the subject. In some embodiments, a dose of the DR IL-18 composition comprises about 15 μg/kg, about 30 μg/kg, about 90 μg/kg, about 180 μg/kg, about 360 μg/kg, about 600 μg/kg, about 900 μg/kg, or about 1200 μg/kg of the polypeptide per body weight of the subject. In some embodiments, a dose of the DR IL-18 composition comprises between about 15 μg/kg to about 30 μg/kg, about 30 μg/kg to about 90 μg/kg, about 90 μg/kg to about 180 μg/kg, about 180 μg/kg to about 360 μg/kg, about 360 μg/kg to about 600 μg/kg, about 600 μg/kg to about 900 μg/kg, or about 900 μg/kg to about 1200 μg/kg of the polypeptide per body weight of the subject. In some embodiments, a subsequent dose of the DR IL-18 composition comprises a greater amount of the polypeptide per body weight of the subject than an amount of the polypeptide administered previously to the subject. In some embodiments, the previous amount of the polypeptide was tolerated by the subject. In some embodiments, a subsequent dose of the DR IL-18 composition comprises a greater amount of the polypeptide per body weight of the subject than an amount of the polypeptide in a previous dose of the DR IL-18 composition. In some embodiments, the previous dose was tolerated by the subject. In some embodiments, a subsequent dose of the DR IL-18 composition comprises a same or similar amount of the polypeptide per body weight of the subject to an amount of the polypeptide administered previously to the subject. In some embodiments, the amount of the polypeptide is associated with a recorded Treatment-Emergent Adverse Event in the subject. In some embodiments, the recorded Treatment-Emergent Adverse Event is a Grade 1 or Grade 2 Treatment-Emergent Adverse Event according to NCI CTCAE Version 5.0. In some embodiments, the recorded Treatment-Emergent Adverse Event is associated with no limitation to mild limitation in activity of the subject. In some embodiments, the recorded Treatment-Emergent Adverse Event is associated with providing no or minimal medical intervention, assistance, or therapy to the subject. In some embodiments, the amount is a highest tolerable amount. In some embodiments, a subsequent dose of the DR IL-18 composition comprises a same or similar amount of the polypeptide per body weight of the subject to an amount of the polypeptide in a previous dose of the DR IL-18 composition. In some embodiments, the previous dose is associated with causing a recorded Treatment-Emergent Adverse Event in the subject. In some embodiments, the recorded Treatment-Emergent Adverse Event is a Grade 1 or Grade 2 Treatment-Emergent Adverse Event according to NCI CTCAE Version 5.0. In some embodiments, the recorded Treatment-Emergent Adverse Event is associated with no limitation to mild limitation in activity of the subject. In some embodiments, the recorded Treatment-Emergent Adverse Event is associated with providing no or minimal medical intervention, assistance, or therapy to the subject. In some embodiments, the previous dose is a highest tolerable dose. In some embodiments, a subsequent dose of the DR IL-18 composition comprises a lesser amount of the polypeptide per body weight of the subject than an amount of the polypeptide administered previously to the subject. In some embodiments, the subject did not tolerate the amount of the polypeptide administered previously. In some embodiments, the amount of the polypeptide administered previously is associated with dose limiting toxicity (DLT). In some embodiments, the subsequent dose is a previously tolerated dose. In some embodiments, a subsequent dose of the DR IL-18 composition comprises a lesser amount of the polypeptide per body weight of the subject than an amount of the polypeptide in a previous dose of the DR IL-18 composition. In some embodiments, the previous dose was not tolerated by the subject. In some embodiments, the previous dose is associated with dose limiting toxicity (DLT). In some embodiments, the subsequent dose is a previously tolerated dose. In some embodiments, the anti-PD-1 and DR IL-18 compositions are administered to the subject is a series of cycles, wherein each cycle comprises about 21 days. In some embodiments, the anti-PD-1 antibody composition is administered once per cycle. In some embodiments, the DR IL-18 composition is administered one, two, or three times during each cycle. In some embodiments, the series of cycles comprises a current cycle and a next cycle, wherein the next cycle runs consecutively to the current cycle. In some embodiments, the series of cycles comprises a first cycle, and wherein the first cycle comprises a first day. In some embodiments, a dose of the anti-PD-1 antibody composition comprising 200 mg of pembrolizumab is administered to the subject on the first day of the first cycle. In some embodiments, a dose of the DR IL-18 composition comprising 30 μg/kg of the polypeptide is administered to the subject on the first day of the first cycle. In some embodiments, the series of cycles comprises at least 8 cycles. In some embodiments, the series of cycles comprises no more than 35 cycles. In some embodiments, the anti-PD-1 and DR IL-18 compositions are administered for at least 24 weeks. In some embodiments, the anti-PD-1 and DR IL-18 compositions are administered until the subject has been administered at least 8 doses of the anti-PD-1 antibody composition. In some embodiments, the anti-PD-1 and DR IL-18 compositions are administered until the subject exhibits a complete response (CR) and has been treated for at least 24 weeks or has been administered at least 8 doses of the anti-PD-1 antibody composition. In some embodiments, the anti-PD-1 and DR IL-18 compositions are administered until the subject exhibits a complete response (CR) and has been treated for at least 8 cycles. In some embodiments, the anti-PD-1 and DR IL-18 compositions are administered until the subject exhibits a complete response (CR) and has been administered at least 8 doses of the anti-PD-1 antibody composition. In some embodiments, the anti-PD-1 and DR IL-18 compositions are administered until the subject has been administered at least about 24 doses of the anti-PD-1 antibody composition. In some embodiments, the anti-PD-1 and DR IL-18 compositions are administered until the subject exhibits an intervention-related toxicity specified as a reason for permanent discontinuation. In some embodiments, the intervention-related toxicity is at least one of a Grade 4, Grade 3, or recurrent Grade 2 Treatment-Emergent Adverse Event according to NCI CTCAE Version 5.0. In some embodiments, the Treatment-Emergent Adverse Event is associated with marked or severe limitation to extreme limitation in activity of the subject. In some embodiments, the Treatment-Emergent Adverse Event is life threatening or requires significant medical intervention or hospitalization. In some embodiments, the anti-PD-1 and DR IL-18 compositions are administered for at most about 2 years. In some embodiments, at most 35 doses of the anti-PD-1 antibody composition are administered to the subject. In some embodiments, a dose of the anti-PD-1 antibody composition is administered to the subject at least about 60 minutes before a dose of the DR IL-18 composition is administered to the subject. In some embodiments, a dose of the DR IL-18 composition is administered to the subject at least about 60 minutes after a dose of the anti-PD-1 antibody composition is administered to the subject. In some embodiments, (a) and (b) are performed sequentially. In some embodiments, (a) is performed before (b). In some embodiments, (b) is performed before (a). In some embodiments, (a) and (b) are performed simultaneously. In some embodiments, a dose of the anti-PD-1 antibody composition is administered to the subject by intravenous infusion. In some embodiments, wherein each dose of the anti-PD-1 antibody composition is administered to the subject by intravenous infusion. In some embodiments, a dose of the DR IL-18 composition is administered to the subject by subcutaneous injection. In some embodiments, each dose of the DR IL-18 composition is administered to the subject by subcutaneous injection. In some embodiments, the disease comprises cancer or a solid tumor. In some embodiments, the cancer or solid tumor is associated with melanoma, non-small cell lung cancer (NSCLC), small cell lung cancer, head and neck squamous cell cancer (HNSCC), classical Hodgkin Lymphoma (cHL), primary mediastinal large B-cell lymphoma (PMBCL), urothelial carcinoma, microsatellite instability-high (MSI-H) or mismatch repair deficient cancer (dMMR) cancer, microsatellite instability-high or mismatch repair deficient colorectal cancer (CRC), colorectal cancer, gastric cancer, esophageal or gastroesophageal junction cancer, esophageal or gastroesophageal junction adenocarcinoma, locally advanced or metastatic esophageal or gastroesophageal junction (GEJ) carcinoma, malignant pleural mesothelioma, cervical cancer, ovarian cancer, hepatocellular carcinoma (HCC), Merkel cell carcinoma (MCC), kidney cancer, renal cell carcinoma (RCC), bladder cancer, endometrial carcinoma, liver cancer, tumor mutational burden-high (TMB-H) cancer, cutaneous squamous cell carcinoma (cSCC), triple-negative breast cancer (TNBC), or any combination thereof. In some embodiments, the cancer or solid tumor is resistant to PD-1 checkpoint inhibitors. In some embodiments, the cancer or solid tumor is associated with melanoma, Merkel cell carcinoma, RCC, urothelial, NSCLC (with no epidermal growth factor receptor, TRK receptor, or anaplastic lymphoma kinase positive mutations/fusions), TNBC, SCCHN, MSI-H, TMB-H or mismatch repair deficient, gastric, cervical, endometrial, cutaneous squamous, small cell lung, esophageal, HCC, or any combination thereof, and wherein the cancer or solid tumor is resistant to PD-1 checkpoint inhibitors. In some embodiments, the cancer or solid tumor is associated with platinum-resistant ovarian cancer or microsatellite stable colorectal cancer.

Another aspect of the present disclosure provides a method of treating a disease in a human patient comprising administering to the patient an anti-PD-1 antibody, or antigen binding fragment thereof, in combination with a decoy-resistant (DR) IL-18 composition comprising a polypeptide to the subject, wherein the polypeptide is a modified IL-18 polypeptide comprising an amino acid sequence set forth in any one of SEQ ID NOs: 36-43, wherein the anti-PD-1 antibody or antigen binding fragment thereof comprises a light chain complementarity determining regions (CDRs) comprising a sequence of amino acids as set forth in SEQ ID NOs: 1, 2 and 3 and heavy chain CDRs comprising a sequence of amino acids as set forth in SEQ ID NOs: 6, 7 and 8.

Another aspect of the present disclosure provides a method of treating a disease in a human patient comprising administering to the patient an anti-PD-1 antibody, or antigen binding fragment thereof, in combination with a decoy-resistant (DR) IL-18 composition comprising a polypeptide comprising an amino acid sequence set forth in SEQ ID NO: 41, wherein the anti-PD-1 antibody or antigen binding fragment thereof comprises a light chain complementarity determining regions (CDRs) comprising a sequence of amino acids as set forth in SEQ ID NOs: 1, 2 and 3 and heavy chain CDRs comprising a sequence of amino acids as set forth in SEQ ID NOs: 6, 7 and 8. In some embodiments, the method further comprises administering CRS prophylaxis to the subject with or before administering the DR IL-18 composition. In some embodiments, the CRS prophylaxis comprises at least one of a NSAID, acetaminophen, diphenhydramine, histamine H1 antagonist, famotidine, H2 blocker, or fluids administered to the subject. In some embodiments, the CRS prophylaxis comprises administering to the subject at least a NSAID and acetaminophen; at least a NSAID and a H1 antagonist; at least acetaminophen and a H1 antagonist; or at least a NSAID, acetaminophen, and a H1 antagonist. In some embodiments, the CRS prophylaxis is administered to the subject orally or intravenously.

In some embodiments, the anti-PD-1 antibody, or antigen binding fragment thereof is an anti-PD-1 monoclonal antibody. In some embodiments, the anti-PD-1 monoclonal antibody is administered to the patient at a dose of about 200 mg once every three weeks. In some embodiments, the anti-PD-1 monoclonal antibody is administered to the patient at a dose of about 400 mg once every six weeks. In some embodiments, the anti-PD-1 monoclonal antibody is administered to the patient by an IV infusion. In some embodiments, the anti-PD-1 monoclonal antibody is administered to the patient by an IV infusion over about 30 minutes on day 1 of each treatment cycle. In some embodiments, the anti-PD-1 antibody, or antigen binding fragment thereof, is pembrolizumab. In some embodiments, the anti-PD-1 monoclonal antibody is pembrolizumab. In some embodiments, the anti-PD-1 monoclonal antibody is a pembrolizumab variant. In some embodiments, the anti-PD-1 antibody or antigen-binding fragment thereof is administered as part of a composition, wherein the composition comprises 130 mg/mL of the anti-PD-1 antibody or antigen-binding fragment thereof. In some embodiments, the anti-PD-1 antibody or antigen-binding fragment thereof is administered as part of a composition, wherein the composition comprises 165 mg/mL of the anti-PD-1 antibody or antigen-binding fragment thereof.

Another aspect of the present disclosure provides for use of an anti-PD-1 antibody, or antigen binding fragment thereof, in a method of treating cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of the invention will be better understood when read in conjunction with the appended drawings. It should be understood that the invention is not limited to the precise arrangements and instrumentalities of the embodiments shown in the drawings.

FIG. 1 depicts substantially reduced CT26 (colorectal) tumor growth in mice treated with a combination of a decoy resistant (DR) IL-18 polypeptide and an anti-PD-1 immune checkpoint inhibitor (ICI) antibody. The y-axis is tumor growth in cubic millimeters (mm3) and error bars represent standard deviation.

FIG. 2 depicts reduced MC38 (colorectal) tumor growth in mice treated with a combination of a DR IL-18 polypeptide and an anti-PD-1 ICI antibody as compared to either DR IL-18 polypeptide or anti-PD-1 ICI monotherapy. The y-axis is tumor growth in mm3 and error bars represent standard deviation. The inset provides a zoom-in of the area of tumor growth between zero and 1000 mm3.

FIG. 3. shows amino acid sequences of the light chain and heavy chain for an exemplary anti-PD-1 monoclonal antibody useful in the invention (SEQ ID NOs: 5 and 10, respectively). Light chain and heavy chain variable regions are underlined (SEQ ID NOs: 4 and 9, respectively) and CDRs are bold.

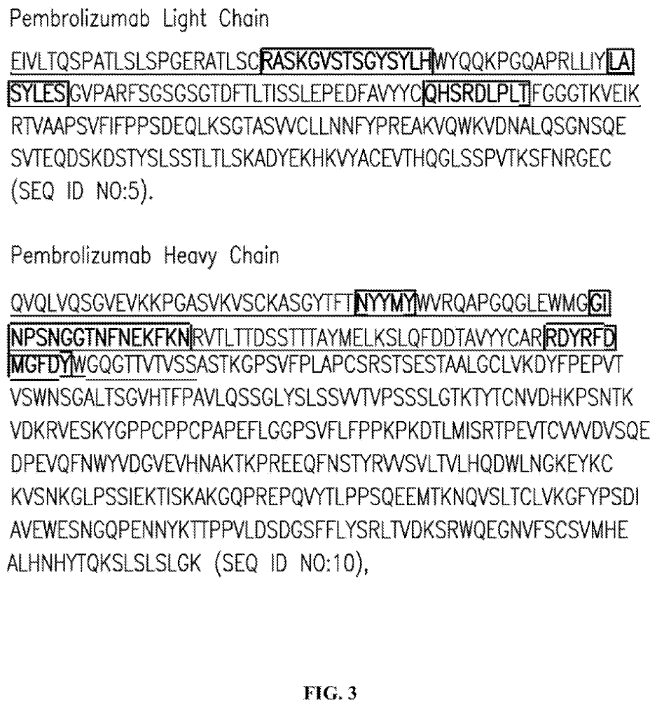

### DETAILED DESCRIPTION

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

As used herein, the terms “at least one” item or “one or more” item each include a single item selected from the list as well as mixtures of two or more items selected from the list.

The terms “administration” or “administer” refers to the act of injecting or otherwise physically delivering a substance as it exists outside the body (e.g., an anti-PD-1 antibody) into a patient or subject, such as by oral, mucosal, intradermal, intravenous, subcutaneous, intramuscular delivery, and/or any other methods of physical delivery described herein or known in the art.

The term “subject” (alternatively “patient”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorph (e.g., rabbits), rodent (e.g., rats or mice), and Procyon lotor (e.g., raccoons). In particular embodiments, the subject is human.

The term “subject in need thereof” as used herein refers to a subject diagnosed with or suspected of having cancer as defined herein.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies, and may include post-translational modifications thereof (e.g., C-terminal Lysine clipping in the heavy chain, conversion of glutamine or glutamic acid to pyroglutamate) that may occur when an antibody is recombinantly expressed in host cells (e.g., CHO cells), or during purification/storage. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, fusion proteins comprising an antigen binding fragment thereof that competes with the intact antibody for specific.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.”

Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. As referred to herein the light chain CDRs are CDRL1, CDRL2 and CDRL3, respectively, and the heavy chain CDRs are CDRH1, CDRH2 and CDRH3, respectively. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} β-sheet framework, or one of three hypervariable regions (L1, L2, or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved β-sheet framework, and thus are able to adapt to different conformations. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

As used herein, unless otherwise indicated, “antibody fragment” or “antigen binding fragment” refers to a fragment of an antibody that retains the ability to bind specifically to the antigen, e.g., fragments that retain one or more CDR regions and the ability to bind specifically to the antigen. An antibody that “specifically binds to” PD-1 is an antibody that exhibits preferential binding to PD-1 (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′) 2, Fd, Fv, fragments including CDRs, and single chain variable fragment antibodies (scFv), and polypeptides that contain at least a portion of an immunoglobulin that is sufficient to confer specific antigen binding to the antigen (e.g., PD-1). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell, for example, a cancer cell.

An “intact” antibody is one comprising an antigen-binding site as well as a constant domain (CL) and at least heavy chain constant regions, CH1, CH2 and CH3. The constant regions may include human constant regions or amino acid sequence variants thereof. In certain embodiments, an intact antibody has one or more effector functions.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. A suitable route and method of administration may vary depending on a number of factors such as the specific therapeutic agent being used, the rate of absorption desired, specific formulation or dosage form used, type or severity of the disorder being treated, the specific site of action, and conditions of the patient, and can be readily selected by a person skilled in the art.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in treatment methods include cytostatic and/or cytotoxic agents.

The term “variant” when used in relation to an antibody (e.g., an anti-PD-1 antibody) or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified anti-PD-1 antibody. Variants may be naturally occurring or may be artificially constructed. Polypeptide variants may be prepared from the corresponding nucleic acid molecules encoding the variants. In specific embodiments, an antibody variant (e.g., an anti-PD-1 antibody variant) at least retains the antibody functional activity. In some embodiments, an anti-PD-1 antibody variant binds to PD-1 and/or is antagonistic to PD-1 activity.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“RECIST 1.1 Response Criteria” as used herein means the definitions set forth in Eisenhauer, E. A. et al., Eur. J. Cancer 45:228-247 (2009) for target lesions or nontarget lesions, as appropriate based on the context in which response is being measured.

“Sustained response” means a sustained therapeutic effect after cessation of treatment as described herein. In some embodiments, the sustained response has a duration that is at least the same as the treatment duration, or at least 1.5, 2.0, 2.5 or 3 times longer than the treatment duration.

“Non-responder patient”, when referring to a specific anti-tumor response to a treatment described herein, means the patient did not exhibit an anti-tumor response.

“Responder patient” when referring to a specific anti-tumor response to a treatment described herein, means the patient exhibited an anti-tumor response.

“Treat” or “treating” cancer as used herein means to administer the agent(s) described (such as e.g., an anti-human PD-1 monoclonal antibody or antigen binding fragment thereof, a decoy resistant (DR) IL-18 composition, combinations thereof, and the like), to a subject having cancer or diagnosed with cancer to achieve at least one positive therapeutic effect, such as, for example, reduced number of cancer cells, reduced tumor size, reduced rate of cancer cell infiltration into peripheral organs, or reduced rate of tumor metastasis or tumor growth, comprising administration by oral, mucosal, intradermal, intravenous, subcutaneous, intramuscular delivery, and/or any other methods of physical delivery described herein or known in the art. Typically, the agent(s) of the treatment method are administered in an amount effective to alleviate one or more disease symptoms in the treated subject or population, whether by inducing the regression of or inhibiting the progression of such symptom(s) by any clinically measurable degree. The amount of the agent(s) of the treatment method that is effective to alleviate any particular disease symptom may vary according to factors such as the disease state, age, and weight of the patient, and the ability of the therapeutic combination to elicit a desired response in the subject. Whether a disease symptom has been alleviated can be assessed by any clinical measurement typically used by physicians or other skilled healthcare providers to assess the severity or progression status of that symptom. “Treatment” may include one or more of the following: inducing/increasing an antitumor immune response, decreasing the number of one or more tumor markers, halting or delaying the growth of a tumor or blood cancer or progression of disease such as cancer, stabilization of disease, inhibiting the growth or survival of tumor cells, eliminating or reducing the size of one or more cancerous lesions or tumors, decreasing the level of one or more tumor markers, ameliorating or abrogating the clinical manifestations of disease, reducing the severity or duration of the clinical symptoms, prolonging the survival or patient relative to the expected survival in a similar untreated patient, and inducing complete or partial remission of a cancerous condition, wherein the disease is cancer.

The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

Positive therapeutic effects in cancer can be measured in a number of ways (See, W. A. Weber, J. Nucl. Med. 50: 1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a therapy of the disclosure is any of partial response (PR), complete response (CR), objective response (OR), progression-free survival (PFS), disease-free survival (DFS), and overall survival (OS). PFS, also referred to as “Time to Tumor Progression” indicates the length of time during and after treatment that the cancer does not grow, and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naive or untreated individuals or patients. In some embodiments, response to a therapy of the disclosure is any of PR, CR, PFS, DFS, or OR that is assessed using RECIST 1.1 response criteria. The treatment regimen for a therapy of the disclosure that is effective to treat a cancer patient may vary according to factors such as the disease state, age, and weight of the patient, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Cytokine Release Syndrome” or CRS, as used herein, refers to an acute systemic inflammatory syndrome that can be triggered by a variety of factors such as infections and may occur after treatment with some types of immunotherapy, such as monoclonal antibodies and chimeric antigen receptor (CAR) T cell therapies, as well as some non-protein-based cancer drugs. CRS is characterized by a large, rapid increase in cytokines and inflammatory response. Signs and symptoms of CRS include fever, fatigue, nausea, headache, rash, arthralgia, myalgia, rapid heartbeat, hypotension, and trouble breathing. CRS can progress to an uncontrolled systemic inflammatory response with vasopressor-requiring circulatory shock, vascular leakage, disseminated intravascular coagulation, and multi-organ system failure. Patients may have a mild reaction, or reactions may be severe or life threatening. Grading of CRS can be performed according to the Consensus American Society for Transplantation and Cellular Therapy (ASTCT) grading system, such as e.g., that described in Lee et al. Biology of Blood and Marrow Transplantation. 25 (2019) 625-639; the disclosure of which is incorporated herein by reference in its entirety.

“PD-1 antagonist” or “anti-PD-1 antibody” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCDIL1, PDL1, B7H1, B7-4, CD274 and B7-H for PD-L1; and PDCDIL2, PDL2, B7-DC, Btdc and CD273 for PD-L2. In any of the treatment methods, medicaments and uses of the disclosure in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP 005009. Human PD-L1 and PD-L2 amino acid sequences can be found in NCBI Locus No.: NP 054862 and NP_079515, respectively.

“Anti-PD-1 antibody composition” means a composition or formulation comprising the anti-PD-1 antibody (for example, pembrolizumab) in a specific concentration that is administered to a human patient or subject.

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 3. Pembrolizumab has been approved by the U.S. FDA as described in the Prescribing Information for KEYTRUDA® (Merck & Co., Inc., Rahway, NJ USA; initial U.S. approval 2014, updated January 2024). The term pembrolizumab includes mAb's having a structure as described above (Id.) but which do not include the C-terminal lysine in the heavy chain.

“Pembrolizumab variant” as used herein means a monoclonal antibody that comprises heavy chain and light chain sequences that are identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residues of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“Platinum-containing chemotherapy” (also known as platins) refers to the use of chemotherapeutic agent(s) used to treat cancer that are coordination complexes of platinum. Platinum-containing chemotherapeutic agents are alkylating agents that crosslink DNA, resulting in ineffective DNA mismatch repair and generally leading to apoptosis. Examples of platins include cisplatin, carboplatin, and oxaliplatin.

### PD-1 Antagonists or Anti-Human PD-1 Monoclonal Antibodies Useful in the Methods of the Disclosure

Examples of mAbs that bind to human PD-1, useful in the treatment methods, compositions, and uses of the disclosure, are described in U.S. Pat. Nos. 7,521,051, 8,008,449, and 8,354,509. Specific anti-human PD-1 mAbs useful as the PD-1 antagonist in the treatment methods, compositions, and uses of the disclosure include: pembrolizumab (formerly known as MK-3475, SCH 900475 and lambrolizumab), a humanized IgG4 mAb with the structure described in WHO Drug Information, Vol. 27, No. 2, pages 161-162 (2013) and which comprises the heavy and light chain amino acid sequences shown in FIG. 7, and the humanized antibodies h409A11, h409A16 and h409A17, which are described in WO 2008/156712.

Provided herein are PD-1 antagonists or anti-human PD-1 monoclonal antibodies that can be used in any of the methods, compositions, kits, and uses disclosed herein, including any chemical compound or biological molecule that blocks binding of PD-L1 to PD-1 and preferably also blocks binding of PD-L2 to PD-1.

In one embodiment, the anti-PD-1 antibody is pembrolizumab. In one embodiment, the anti-human PD-1 monoclonal antibody is pembrolizumab.

In some embodiments, an anti-human PD-1 antibody or antigen binding fragment thereof for use in the methods and uses of the disclosure comprises three light chain CDRs of CDRL1, CDRL2 and CDRL3 and/or three heavy chain CDRs of CDRH1, CDRH2 and CDRH3.

In one embodiment, CDRL1 has the amino acid sequence as set forth in SEQ ID NO:1 or a variant of the amino acid sequence as set forth in SEQ ID NO:1, CDRL2 has the amino acid sequence as set forth in SEQ ID NO:2 or a variant of the amino acid sequence as set forth in SEQ ID NO:2, and CDRL3 has the amino acid sequence as set forth in SEQ ID NO:3 or a variant of the amino acid sequence as set forth in SEQ ID NO:3.

In one embodiment, CDRH1 has the amino acid sequence as set forth in SEQ ID NO:6 or a variant of the amino acid sequence as set forth in SEQ ID NO:6, CDRH2 has the amino acid sequence as set forth in SEQ ID NO:7 or a variant of the amino acid sequence as set forth in SEQ ID NO:7, and CDRH3 has the amino acid sequence as set forth in SEQ ID NO:8 or a variant of the amino acid sequence as set forth in SEQ ID NO:8.

In one embodiment, the three light chain CDRs have the amino acid sequences as set forth in SEQ ID NO:1, SEQ ID NO:2, and SEQ ID NO:3 and the three heavy chain CDRs have the amino acid sequences as set forth in SEQ ID NO:6, SEQ ID NO:7 and SEQ ID NO:8.

In one embodiment, the three light chain CDRs have the amino acid sequences as set forth in SEQ ID NO:1, SEQ ID NO:2, and SEQ ID NO:3 and the three heavy chain CDRs have the amino acid sequences as set forth in SEQ ID NO:6, SEQ ID NO:7 and SEQ ID NO:8.

In a further embodiment, CDRL1 has the amino acid sequence as set forth in SEQ ID NO: 21 or a variant of the amino acid sequence as set forth in SEQ ID NO:21, CDRL2 has the amino acid sequence as set forth in SEQ ID NO:22 or a variant of the amino acid sequence as set forth in SEQ ID NO:22, and CDRL3 has the amino acid sequence as set forth in SEQ ID NO:23 or a variant of the amino acid sequence as set forth in SEQ ID NO:23.

In yet another embodiment, CDRH1 has the amino acid sequence as set forth in SEQ ID NO: 24 or a variant of the amino acid sequence as set forth in SEQ ID NO:24, CDRH2 has the amino acid sequence as set forth in SEQ ID NO: 25 or a variant of the amino acid sequence as set forth in SEQ ID NO:25, and CDRH3 has the amino acid sequence as set forth in SEQ ID NO: 26 or a variant of the amino acid sequence as set forth in SEQ ID NO:26.

In another embodiment, the three light chain CDRs have the amino acid sequences as set forth in SEQ ID NO:21, SEQ ID NO:22, and SEQ ID NO:23 and the three heavy chain CDRs have the amino acid sequences as set forth in SEQ ID NO:24, SEQ ID NO:25 and SEQ ID NO: 26.

Some anti-human PD-1 antibody and antigen binding fragments comprise a light chain variable region and a heavy chain variable region. In some embodiments, the light chain variable region comprises the amino acid sequence as set forth in SEQ ID NO:4 or a variant of the amino acid sequence as set forth in SEQ ID NO:4, and the heavy chain variable region comprises the amino acid sequence as set forth in SEQ ID NO:9 or a variant of the amino acid sequence as set forth in SEQ ID NO:9. In further embodiments, the heavy chain variable region comprises the amino acid sequence as set forth in SEQ ID NO:27 or a variant of the amino acid sequence as set forth in SEQ ID NO:27 and the light chain variable region comprises the amino acid sequence as set forth in SEQ ID NO:28 or a variant of the amino acid sequence as set forth in SEQ ID NO: 28, the amino acid sequence as set forth in SEQ ID NO:29 or a variant of the amino acid sequence as set forth in SEQ ID NO:29, or the amino acid sequence as set forth in SEQ ID NO: 30 or a variant of the amino acid sequence as set forth in SEQ ID NO:30. In such embodiments, a light chain variable region or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In one embodiment of the methods, kits or uses of the disclosure, the anti-human PD-1 antibody or antigen binding fragment comprises a light chain variable region comprising or consisting of the amino acid sequence as set forth in SEQ ID NO:4 and a heavy chain variable region comprising or consisting of the amino acid sequence as set forth in SEQ ID NO:9. In one embodiment, the anti-human PD-1 antibody or antigen binding fragment comprises a light chain variable region comprising or consisting of the amino acid sequence as set forth in SEQ ID NO: 28 and a heavy chain variable region comprising or consisting of the amino acid sequence as set forth in SEQ ID NO:27. In a further embodiment, the anti-human PD-1 antibody or antigen binding fragment comprises a light chain variable region comprising or consisting of the amino acid sequence as set forth in SEQ ID NO:29 and a heavy chain variable region comprising or consisting of the amino acid sequence as set forth in SEQ ID NO:27. In another embodiment, the antibody or antigen binding fragment comprises a light chain variable region comprising or consisting of the amino acid sequence as set forth in SEQ ID NO:30 and a heavy chain variable region comprising or consisting of the amino acid sequence as set forth in SEQ ID NO:27.

In another embodiment, the methods, kits or uses of the disclosure comprise an anti-human PD-1 antibody or antigen binding protein that has a V_{L} domain and/or a V_{H} domain with at least 99%, 98%, 97%, 96%, 95%, 90%, 85%, 80%, 75% or 50% sequence homology to one of the V_{L} domains or V_{H} domains described above, and exhibits specific binding to PD-1. In another embodiment, the anti-human PD-1 antibody or antigen binding protein of the methods comprises V_{L} and V_{H} domains having up to 1, 2, 3, 4, or 5 or more amino acid substitutions, and exhibits specific binding to PD-1.

In any of the embodiments above, the PD-1 antagonist may be a full-length anti-PD-1 antibody or an antigen binding fragment thereof that specifically binds human PD-1. In certain embodiments, the PD-1 antagonist is a full-length anti-PD-1 antibody selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Different constant domains may be appended to the V_{L} and V_{H} regions provided herein. For example, if a particular intended use of an antibody (or fragment) were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

In some embodiments, the PD-1 antagonist is an anti-PD-1 antibody comprising a light chain comprising or consisting of a sequence of amino acid residues as set forth in SEQ ID NO: 5 and a heavy chain comprising or consisting of a sequence of amino acid residues as set forth in SEQ ID NO:10. In further embodiments, the PD-1 antagonist is an anti-PD-1 antibody comprising a light chain comprising or consisting of a sequence of amino acid residues as set forth in SEQ ID NO:32 and a heavy chain comprising or consisting of a sequence of amino acid residues as set forth in SEQ ID NO:31. In additional embodiments, the PD-1 antagonist is an anti-PD-1 antibody comprising a light chain comprising or consisting of a sequence of amino acid residues as set forth in SEQ ID NO:33 and a heavy chain comprising or consisting of a sequence of amino acid residues as set forth in SEQ ID NO:31. In yet additional embodiments, the PD-1 antagonist is an anti-PD-1 antibody comprising a light chain comprising or consisting of a sequence of amino acid residues as set forth in SEQ ID NO:34 and a heavy chain comprising or consisting of a sequence of amino acid residues as set forth in SEQ ID NO:31. In some methods, the PD-1 antagonist is pembrolizumab or a pembrolizumab biosimilar.

Ordinarily, amino acid sequence variants of the anti-PD-1 antibodies and antigen binding fragments will have an amino acid sequence having at least 75% amino acid sequence identity with the amino acid sequence of a reference antibody or antigen binding fragment (e.g. heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the anti-PD-1 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Likewise, either class of light chain can be used in the compositions and methods herein. Specifically, kappa, lambda, or variants thereof are useful in the present compositions and methods.

TABLE 3
Exemplary PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHWY | 4 |
| Region | QQKPGQAPRLLIYLASYLESGVPARFSGSGSGTDFTLTISS | |
| | LEPEDFAVYYCQHSRDLPLTFGGGTKVEIK | |
| Light Chain | EIVLTQSPATLSLSPGERATLSCRASKGVSTSGYSYLHWY | 5 |
| | QQKPGQAPRLLIYLASYLESGVPARFSGSGSGTDFTLTISS | |
| | LEPEDFAVYYCQHSRDLPLTFGGGTKVEIKRTVAAPSVFI | |
| | FPPSDEQLKSGTASVVCLLNNFYPREAKVQWKVDNALQ | |
| | SGNSQESVTEQDSKDSTYSLSSTLTLSKADYEKHKVYAC | |
| | EVTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYWV | 9 |
| Region | RQAPGQGLEWMGGINPSNGGTNFNEKFKNRVTLTTDSST | |
| | TTAYMELKSLQFDDTAVYYCARRDYRFDMGFDYWGQG | |
| | TTVTVSS | |
| Heavy | QVQLVQSGVEVKKPGASVKVSCKASGYTFTNYYMYWV | 10 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLGK | |

TABLE 4
Additional PD-1 Antibodies and Antigen Binding Fragments Useful in the
Methods and Uses of the Disclosure.
A. Antibodies and antigen binding fragments comprising light and heavy chain CDRs of
hPD-1.08A in WO2008/156712

| | | |
|---|---|---|
| CDRL1 | RASKSVSTSGFSYLH | SEQ ID NO: 21 |
| CDRL2 | LASNLES | SEQ ID NO: 22 |
| CDRL3 | QHSWELPLT | SEQ ID NO: 23 |
| CDRH1 | SYYLY | SEQ ID NO: 24 |
| CDRH2 | GVNPSNGGTNFSEKFKS | SEQ ID NO: 25 |
| CDRH3 | RDSNYDGGFDY | SEQ ID NO: 26 |

B. Antibodies and antigen binding fragments comprising the mature h109A heavy chain
variable region and one of the mature K09A light chain variable regions in WO
2008/156712

| | | |
|---|---|---|
| Heavy | QVQLVQSGVEVKKPGASVKVSCKASGYTFTNYYMYWVR | SEQ ID NO: 27 |
| chain VR | QAPGQGLEWMGGINPSNGGTNFNEKFKNRVTLTTDSSTTT | |
| | AYMELKSLQFDDTAVYYCARRDYRFDM | |
| | GFDYWGQGTTVTVSS | |
| Light | EIVLTQSPATLSLSPGERATLSCRASKGVSTSGYSYLHWYQ | SEQ ID NO: 28 |
| chain VR | QKPGQAPRLLIYLASYLESGVPARFSGSGSGTDFTLTISSLE | or |
| | PEDFAVYYCQHSRDLPLTFGGGTKVEIK | |
| | EIVLTQSPLSLPVTPGEPASISCASKGVSTSGYSYLHWYLQ | SEQ ID NO: 29 |
| | KPGQSPQLLIYLASYLESGVPDRFSGSGSGTDFTLKISRVEA | or |
| | EDVGVYYCHSRDLPLTFGQGTKLEIK | |
| | DIVMTQTPLSLPVTPGEPASISCASKGVSTSGYSYLHWYLQ | SEQ ID NO: 30 |
| | KPGQSPQLLIYLASYLESGVPDRFSGSGSGTAFTLKISRVEA | |
| | EDVGLYYCQHSRDLPLTFGQGTKLEIK | |

C. Antibodies and antigen binding fragments comprising the mature 409 heavy chain and
one of the mature K09A light chains in WO 2008/156712

| | | |
|---|---|---|
| Heavy | QVQLVQSGVEVKKPGASVKVSCASGYTFTNYYMWVRQA | SEQ ID NO: 31 |
| chain | PGQGLEWMGGINPSNGGTNFNEKFKNRVTLTTDSSTTTAY | |
| | MELKSLQFDDTAVYYCARRDYRFDMFDYWGQGTTVTVS | |
| | SASTKGPSVFPLAPCSRSTSESTAALGCLVKDYFPEPVTVS | |
| | WNSGALTSGVHTFPAVLQSSGLYSLSSVVTVPSSSLGTKTY | |
| | TCNVDHKPSNTKVDKRVESKYGPPCPPCPAPEFLGGPSV | |
| | FLFPPKPKDTLMSRTPEVTCVVDVSQEDPEVQFNWYVDG | |
| | VEVHNAKTKPREEQFNSTYRVVSVLTVLHQDWLNGKEYK | |
| | CKVSNKGLPSSIEKTISKAKGQPREPQVYTLPPSQEEM | |
| | TKNQVSLTCLVKGFYPSDIAVEWESNGQPENNYKTTPPVL | |
| | DSDGSFFLYSRLTVDKSRWQEGNVFSCSVMHEALHNHYT | |
| | QKSLSLSLGK | |
| Light | EIVLTQSPATLSLSPGERATLSCRASKGVSTSGYSYLHWYQ | SEQ ID NO: 32 |
| chain | QKPGQAPRLLIYLASYLESGVPARFSGSGSGTDFTLTISSLE | or |
| | PEDFAVYYCQHSRDLPLTFGGGTKVEIKRTVAAPSVFIFPP | |
| | SDEQLKSGTASVVCLLNNFYPREAKVQWKVDNALQSGNS | |
| | QESVTEQDSKDSTYSLSSTLTLSKADYEKHKVYAC | |
| | EVTHQGLSSPVTKSFNRGEC | |
| | EIVLTQSPLSLPVTPGEPASISCRASKGVSTSGYSYLHWYLQ | SEQ ID NO: 33 |
| | KPGQSPQLLIYLASYLESGVPDRFSGSGSGTDFTLKISRVEA | or |
| | EDVGVYYCHSRDLPLTFGQGTKLEIKRTVAAPSVFIFPPSD | |
| | EQLKSGTASVVCLNNFYPREAKVQWKVDNALQSGNSQES | |
| | VTEQDSKDSTYSLSSTLTLSKADYEKHKVYACETHQGLSS | |
| | PVTKSFNRGEC | |
| | DIVMTQTPLSLPVTPGEPASISCRASKGVSTSGYSYLHWYL | SEQ ID NO: 34 |
| | QKPGQSPQLLIYLASYLESGVPDRFSGSGSGTAFTLKISRVE | |
| | AEDVGLYYC | |
| | QHSRDLPLTFGQGTKLEIKRTVAAPSVFIFPPSDEQ | |
| | LKSGTASVVCLLNNFYPREAKVQWKVDNALQSGNSQESV | |
| | TEQDSKDSTYSLSSTLTLSKADYEKHKVYACEVTHQGLSS | |
| | PVTKSFNRGEC | |

### Anti-PD-1 Antibody Dosing

In some embodiments, the anti-PD-1 antibody (e.g., anti-PD-1 monoclonal antibody) or antigen binding fragment thereof is administered subcutaneously or intravenously, on a weekly, biweekly, triweekly, every 4 weeks, every 5 weeks, every 6 weeks, monthly, bimonthly, or quarterly basis at about 10, about 20, about 50, about 80, about 100, about 200, about 300, about 400, about 500, about 1000 or about 2500 mg/subject.

In some specific methods, the dose of the anti-PD-1 antibody (e.g., anti-PD-1 monoclonal antibody) or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, or from about 2.0 mg/kg to about 4.0 mg/kg.

In some specific methods, the dose of the anti-PD-1 antibody (e.g., anti-PD-1 monoclonal antibody) or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, or from about 200 mg to about 240 mg. In some embodiments, the dose of the anti-PD-1 antibody (e.g., anti-PD-1 monoclonal antibody) or antigen binding fragment thereof is about 50 mg, about 75 mg, about 100 mg, about 125 mg, about 150 mg, about 175 mg, about 200 mg, about 225 mg, about 240 mg, about 250 mg, about 300 mg, about 400 mg, or about 500 mg.

In another embodiment, the PD-1 antagonist in the therapy is pembrolizumab, or a pembrolizumab variant, which is administered in a liquid medicament at a dose selected from the group consisting of 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, or 10 mg/kg Q3W.

In other embodiments, the PD-1 antagonist in the therapy is pembrolizumab, or a pembrolizumab variant, which is administered in a liquid medicament at a flat dose such as 200 mg Q3W or 400 mg Q6W.

In other embodiments, the PD-1 antagonist in the therapy is pembrolizumab which is administered in a liquid medicament at a flat dose such as 200 mg Q3W or 400 mg Q6W.

In some embodiments of the methods, compositions, kits and uses described herein, the anti-human PD-1 antibody (e.g., anti-PD-1 monoclonal antibody) or antigen binding fragment thereof is pembrolizumab, and the human patient is administered about 200 mg, about 240 mg, about 400 mg, about 480 mg, or about 2 mg/kg pembrolizumab once every three or six weeks. In one embodiment, the human patient is administered about 200 mg pembrolizumab once every three weeks. In one embodiment, the human patient is administered about 240 mg pembrolizumab once every three weeks. In one embodiment, the human patient is administered 2 mg/kg pembrolizumab once every three weeks. In one embodiment, the human patient is administered 400 mg pembrolizumab once every six weeks.

In certain embodiments of the methods, compositions, kits and uses described herein, the anti-human PD-1 monoclonal antibody or antigen binding fragment thereof is pembrolizumab and the human patient is administered 200 mg pembrolizumab once every three weeks.

In certain embodiments of the methods, compositions, kits and uses described herein, the anti-human PD-1 monoclonal antibody or antigen binding fragment thereof is pembrolizumab and the human patient is administered 400 mg pembrolizumab once every six weeks.

In some embodiments of the methods, compositions, kits and uses described herein, the anti-human PD-1 monoclonal antibody or antigen binding fragment thereof is pembrolizumab, and the human patient is administered about 200 mg, about 240 mg, about 400 mg, about 480 mg, or about 2 mg/kg pembrolizumab once every six weeks.

In one embodiment, the human patient is administered about 200 mg pembrolizumab once every three weeks. In one embodiment, the human patient is administered about 400 mg pembrolizumab once every six weeks. In one embodiment, the human patient is administered 2 mg/kg pembrolizumab once every three weeks.

In some embodiments, pembrolizumab is provided as a liquid medicament that comprises 25 mg/ml pembrolizumab, 7% (w/v) sucrose, 0.02% (w/v) polysorbate 80 in 10 mM histidine buffer pH 5.5. In other embodiments, pembrolizumab is provided as a liquid medicament that comprises about 125 to about 200 mg/mL of pembrolizumab, or an antigen binding fragment thereof; about 10 mM histidine buffer; about 10 mM L-methionine, or a pharmaceutically acceptable salt thereof; about 7% (w/v) sucrose; and about 0.02% (w/v) polysorbate 80.

In certain embodiments of the methods, compositions, kits and uses described herein, the anti-human PD-1 monoclonal antibody is pembrolizumab, and the human patient is administered about 200 mg pembrolizumab once every three weeks. In certain embodiments of the methods, compositions, kits and uses described herein, the anti-human PD-1 monoclonal antibody is pembrolizumab, and the human patient is administered about 400 mg pembrolizumab once every six weeks.

In some embodiments, the selected dose of pembrolizumab is administered by IV infusion. In one embodiment, the selected dose of pembrolizumab is administered by IV infusion over a time period of between 25 and 40 minutes, or about 30 minutes. In other embodiments, the selected dose of pembrolizumab is administered by subcutaneous injection.

In some embodiments, the selected dose of pembrolizumab is administered subcutaneously. In some embodiments, the amount of pembrolizumab administered subcutaneously to the patient is from 320 mg to 420 mg, from 340 mg to 420 mg, from 345 mg to 415 mg, from 350 mg to 410 mg, from 355 mg to 405 mg, from 360 mg to 400 mg, from 365 mg to 395 mg, from 370 mg to 390 mg, from 375 mg to 385 mg, or from 379 mg to 381 mg. In one embodiment, pembrolizumab is administered by subcutaneous injection at a dose of about 280 mg to about 450 mg. In a further embodiment, pembrolizumab is administered by subcutaneous injection at a dose of about 300 mg to about 450 mg. In yet a further embodiment, pembrolizumab is administered subcutaneously at a dose of about 320 mg to about 450 mg.

In some embodiments, pembrolizumab is administered subcutaneously to the patient, wherein the pembrolizumab is part of a composition and is present in the composition at a concentration of 130 mg/mL. In some embodiments, pembrolizumab administered subcutaneously to the patient, wherein the pembrolizumab is part of a composition and is present in the composition at a concentration of 165 mg/mL. In some embodiments, pembrolizumab is administered subcutaneously to the patient in two injections. In some embodiments, the amount of pembrolizumab administered subcutaneously to the patient is 380 mg in one pre-filled syringe. In some embodiments, the amount of pembrolizumab administered subcutaneously to the patient is 380 mg in two pre-filled syringes. In some embodiments, the amount of pembrolizumab administered subcutaneously to the patient is 395 mg in one pre-filled syringe. In some embodiments, the amount of pembrolizumab administered subcutaneously to the patient is 395 mg in two pre-filled syringes.

In one embodiment, the selected dose of pembrolizumab is administered by subcutaneous injection at a dose that is at least about 1.6 times higher than a 200 mg or a 2 mg/kg dose. In one embodiment, the subcutaneous dose is administered once every three weeks. In one embodiment, the subcutaneous dose is administered once every six weeks. In one embodiment, the bioavailability of the pembrolizumab subcutaneous dose is at least 63%. In one embodiment, the bioavailability of the pembrolizumab subcutaneous dose is at least 64%. In one embodiment, the bioavailability of the pembrolizumab subcutaneous dose is at least 66%.

### Decoy Resistant (DR) Interleukin 18 (IL-18) Polypeptides and DR IL-18 Compositions

The polypeptide of the decoy resistant (DR) IL-18 composition of the present disclosure is a “decoy-resistant” variant of IL-18, designed to be impervious to the decoy receptor IL-18BP, which blocks IL-18 from interacting with its receptor, thereby blocking the cytokine's immunostimulatory activity. The decoy resistant IL-18 polypeptide has been shown in preclinical studies to maintain strong immune stimulation in the tumor microenvironment and is currently in Phase 1a/2 clinical development as a monotherapy in solid tumors.

In some embodiments, the decoy-resistant (DR) IL-18 polypeptide comprises mutations relative to wild-type (WT) IL-18, such as WT IL-18 SEQ ID NO: 35. In some embodiments, the decoy-resistant IL-18 polypeptide comprises at least about two mutations, at least about three mutations, at least about four mutations, at least about five mutations, at least about six mutations, at least about seven mutations, at least about eight mutations, at least about nine mutations, or at least about ten mutations, relative to WT IL-18 as set forth in SEQ ID NO: 35. In some embodiments, the decoy-resistant IL-18 polypeptide comprises at least one mutation (e.g., at least 2, at least 3, at least 4, at least 5, at least 6, at least 7, or 8 mutations) selected from the group consisting of M51K, K53S, Q56L, P57A, M60L, S105D, D110S, and N111R, relative to WT IL-18 as set forth in SEQ ID NO: 35.

In some embodiments, a decoy-resistant IL-18 polypeptide comprises one or more mutations at amino acid positions M51K, K53S, Q56L, P57A, M60L, S105D, D110S, and N111R, relative to WT IL-18 as set forth in SEQ ID NO: 35. In one embodiment, the amino acid sequence of the DR IL-18 polypeptide is

(SEQ ID NO: 36)
YFGKLESKLSVIRNLNDQVLFIDQGNRPLFEDMTDSDCRDNAPRTIFIISKYSDSLARGLA
VTISVKCEKISTLSCENKIISFKEMNPPDNIKDTKSDIIFFQRDVPGHSRKMQFESSSYEGY
FLACEKERDLFKLILKKEDELGDRSIMFTVQNED.

In some embodiments, the amino acid sequence of the DR IL-18 polypeptide comprises 90% or more identity to SEQ ID NO: 36. In some embodiments, the amino acid sequence of the DR IL-18 polypeptide comprises 90-99.5% identity to SEQ ID NO: 36.

In some embodiments, the decoy-resistant IL-18 polypeptide comprises a mutation at amino acid position C38, relative to WT IL-18 as set forth in SEQ ID NO: 35. In some embodiments, the decoy-resistant IL-18 polypeptide comprises a mutation at amino acid position C68, relative to WT IL-18 as set forth in SEQ ID NO: 35. In some embodiments, the decoy-resistant IL-18 polypeptide comprises mutations at amino acid positions C38 and C68, relative to WT IL-18 as set forth in SEQ ID NO: 35.

In some embodiments, the mutation at amino acid position C38 is a substitution mutation. In some embodiments, the mutation at amino acid position C38 is a C38S substitution, relative to WT IL-18 as set forth in SEQ ID NO: 35.

In some embodiments, the mutation at amino acid position C68 is a substitution mutation. In some embodiments, the mutation at amino acid position C68 is a C68S substitution, a C68G substitution, a C68A substitution, a C68V substitution, a C68D substitution, a C68E substitution, or a C68N substitution, relative to WT IL-18 as set forth in SEQ ID NO: 35.

In some embodiments, a decoy-resistant IL-18 polypeptide comprises (i) one or more mutations at amino acid positions M51, K53, Q56, P57, M60, S105, D110, and N111, relative to WT IL-18 as set forth in SEQ ID NO: 35; and (ii) one or more mutations at amino acid positions C38 and C68, relative to WT IL-18 as set forth in SEQ ID NO: 35.

In some embodiments, a decoy-resistant IL-18 polypeptide comprises mutations at amino acid positions M51K, K53S, Q56L, P57A, M60L, S105D, D110S, N111R, C38S and C68S relative to WT IL-18 as set forth in SEQ ID NO: 35. In one embodiment, the amino acid sequence of the DR IL-18 polypeptide is:

(SEQ ID NO: 37)
YFGKLESKLSVIRNLNDQVLFIDQGNRPLFEDMTDSDSRDNAPRTIFIISKYSDSLARGLA
VTISVKSEKISTLSCENKIISFKEMNPPDNIKDTKSDIIFFQRDVPGHSRKMQFESSSYEGY
FLACEKERDLFKLILKKEDELGDRSIMFTVQNED.

In some embodiments, a decoy-resistant IL-18 polypeptide comprises mutations at amino acid positions M51K, K53S, Q56L, P57A, M60L, S105D, D110S, N111R, C38S and C68G relative to WT IL-18 as set forth in SEQ ID NO: 35. In one embodiment, the amino acid sequence of the DR IL-18 polypeptide is:

(SEQ ID NO: 38)
YFGKLESKLSVIRNLNDQVLFIDQGNRPLFEDMTDSDSRDNAPRTIFIISKYSDSLARGLA
VTISVKGEKISTLSCENKIISFKEMNPPDNIKDTKSDIIFFQRDVPGHSRKMQFESSSYEGY
FLACEKERDLFKLILKKEDELGDRSIMFTVQNED.

In some embodiments, a decoy-resistant IL-18 polypeptide comprises mutations at amino acid positions M51K, K53S, Q56L, P57A, M60L, S105D, D110S, N111R, C38S and C68A relative to WT IL-18 as set forth in SEQ ID NO: 35. In one embodiment, the amino acid sequence of the DR IL-18 polypeptide is:

(SEQ ID NO: 39)
YFGKLESKLSVIRNLNDQVLFIDQGNRPLFEDMTDSDSRDNAPRTIFIISKYSDSLARGLA
VTISVKAEKISTLSCENKIISFKEMNPPDNIKDTKSDIIFFQRDVPGHSRKMQFESSSYEGY
FLACEKERDLFKLILKKEDELGDRSIMFTVQNED.

In some embodiments, a decoy-resistant IL-18 polypeptide comprises mutations at amino acid positions M51K, K53S, Q56L, P57A, M60L, S105D, D110S, N111R, C38S and C68V relative to WT IL-18 as set forth in SEQ ID NO: 35. In one embodiment, the amino acid sequence of the DR IL-18 polypeptide is:

(SEQ ID NO: 40)
YFGKLESKLSVIRNLNDQVLFIDQGNRPLFEDMTDSDSRDNAPRTIFIISKYSDSLARGLA
VTISVKVEKISTLSCENKIISFKEMNPPDNIKDTKSDIIFFQRDVPGHSRKMQFESSSYEGY
FLACEKERDLFKLILKKEDELGDRSIMFTVQNED.

In some embodiments, a decoy-resistant IL-18 polypeptide comprises mutations at amino acid positions M51K, K53S, Q56L, P57A, M60L, S105D, D110S, N111R, C38S and C68D relative to WT IL-18 as set forth in SEQ ID NO: 35. In one embodiment, the amino acid sequence of the DR IL-18 polypeptide is:

(SEQ ID NO: 41)
YFGKLESKLSVIRNLNDQVLFIDQGNRPLFEDMTDSDSRDNAPRTIFIISKYSDSLARGLA
VTISVKDEKISTLSCENKIISFKEMNPPDNIKDTKSDIIFFQRDVPGHSRKMQFESSSYEGY
FLACEKERDLFKLILKKEDELGDRSIMFTVQNED.

In some embodiments, a decoy-resistant IL-18 polypeptide comprises mutations at amino acid positions M51K, K53S, Q56L, P57A, M60L, S105D, D110S, N111R, C38S and C68E relative to WT IL-18 as set forth in SEQ ID NO: 35. In one embodiment, the amino acid sequence of the DR IL-18 polypeptide is:

(SEQ ID NO: 42)
YFGKLESKLSVIRNLNDQVLFIDQGNRPLFEDMTDSDSRDNAPRTIFIISKYSDSLARGLA
VTISVKEEKISTLSCENKIISFKEMNPPDNIKDTKSDIIFFQRDVPGHSRKMQFESSSYEGY
FLACEKERDLFKLILKKEDELGDRSIMFTVQNED.

In some embodiments, a decoy-resistant IL-18 polypeptide comprises mutations at amino acid positions M51K, K53S, Q56L, P57A, M60L, S105D, D110S, N111R, C38S and C68N relative to WT IL-18 as set forth in SEQ ID NO: 35. In one embodiment, the amino acid sequence of the DR IL-18 polypeptide is:

(SEQ ID NO: 43)
YFGKLESKLSVIRNLNDQVLFIDQGNRPLFEDMTDSDSRDNAPRTIFIISKYSDSLARGLA
VTISVKNEKISTLSCENKIISFKEMNPPDNIKDTKSDIIFFQRDVPGHSRKMQFESSSYEGY
FLACEKERDLFKLILKKEDELGDRSIMFTVQNED.

In some embodiments, the amino acid sequence of the DR IL-18 polypeptide comprises 90% or more identity to any one of SEQ ID NOs: 36-43. In some embodiments, the amino acid sequence of the DR IL-18 polypeptide comprises 90-99.5% identity to any one of SEQ ID NOs: 36-43. In some embodiments, the amino acid sequence of the DR IL-18 polypeptide comprises 95-99.5% identity to any one of SEQ ID NOs: 36-43. In some embodiments, the amino acid sequence of the DR IL-18 polypeptide comprises 97-99.5% identity to any one of SEQ ID NOs: 36-43. In some embodiments, the amino acid sequence of the DR IL-18 polypeptide comprises 98-99.5% identity to any one of SEQ ID NOs: 36-43.

### Methods

Methods for treating a disease in a subject in need thereof are provided. In some embodiments, the disease is cancer. In some embodiments, the cancer is a solid tumor, such as but not limited to, e.g., a melanoma, Merkel cell, renal cell carcinoma (RCC), urothelial, non-small cell lung cancer (NSCLC), triple-negative breast cancer (TNBC), squamous cell carcinoma of head and neck (SCCHN), any microsatellite instability high (MSI-H), any high tumor mutation burden (TMB-H) or mismatch repair deficient, gastric, cervical, endometrial, cutaneous squamous, small cell lung, esophageal, hepatocellular carcinoma cancer (HCC), including immune checkpoint inhibitor (ICI) resistant (such as e.g., PD-1 checkpoint inhibitor resistant) forms thereof, or platinum resistant ovarian cancer, or microsatellite stable colorectal cancer/tumor. In some embodiments, the cancer is a hematologic cancer, such as but not limited to, e.g., myeloma, B cell lymphoma, or acute myeloid leukemia. One aspect of the present disclosure provides methods of treating the disease in the subject in need thereof comprising administering successive doses of an anti-PD-1 antibody composition comprising pembrolizumab to the subject and administering successive doses of a decoy-resistant (DR) IL-18 composition comprising a polypeptide to the subject, wherein the polypeptide is a modified IL-18 polypeptide comprising an amino acid sequence set forth in any one of SEQ ID NOs: 36-43. Administering such a combination therapy of pembrolizumab and DR IL-18 polypeptide will thereby cause one or more improvements in the condition of the subject, such as e.g., stable disease, immunotherapy-induced regression of the disease, partial response, complete response, or any combination thereof, in the subject.

Provided is a method of combination therapy that includes treating a subject with cancer, the method comprising administering successive doses of a pembrolizumab-containing composition and successive doses of a DR IL-18-containing composition to the subject, wherein the combination therapy causes one or more improvements in the condition of the subject, such as but not limited to e.g., an objective response, a partial response, a complete response, immunotherapy-induced regression of the cancer or tumor, stabilization of disease (e.g., stable disease for at least 12 months), or the like. Administering such a combination therapy of pembrolizumab and DR IL-18 polypeptide will thereby cause an improvement in the condition of the subject that is not obtained through administration of either monotherapy alone and/or is greater than an improvement obtained in a corresponding outcome through administration of pembrolizumab or DR IL-18 polypeptide monotherapy.

Observed improvements in the condition of a subject treated according to the methods described herein will vary and may include any result of treating a disease or treating cancer, including any result of treating a disease or treating cancer described herein. For example, the subject may experience an improvement in one or more symptoms of the condition, e.g., improvement in one or more clinical symptoms of the subject's cancer. In some instances, a subject may experience a reduction in the rate of tumor growth, a reduction in the number of tumors, a reduction in the size of one or more tumors in the subject, a reduction in the clinical stage of a tumor, combinations thereof, or the like.

In some instances, the subject may experience a stabilization of disease, such as e.g., a stabilization of the subject's cancer for some period of time, such as e.g., at least 6 months, at least 12 months, or more. A stable disease is a disease that does not progress, such as e.g., a tumor that does not show substantial growth, as measured using appropriate methods such as computed tomography (CT) or magnetic resonance imaging (MRI) scans, over a predetermined period of time or between two or more relevant timepoints, such as e.g., the start of treatment and 6 months or 12 months after the start of treatment. Stable disease will also include improvements in a subject's disease that to not reach the level or criteria for a partial response (PR).

In some instances, a subject may achieve a PR, where the criteria for a PR, as used herein with respect to a cancer or a tumor, is defined, consistent with RECIST guideline (version 1.1), as at least a 30% decrease in the sum of diameters of target lesions, taking as reference the baseline sum diameters. See e.g., Eisenhauer et al. (2009) European Journal of Cancer 45:228-247, the disclosure of which is incorporated herein by reference in its entirety.

In some instances, a subject may achieve a complete response (CR), where the criteria for a CR, as used herein with respect to a cancer or a tumor, is defined, consistent with RECIST guideline (version 1.1), as disappearance of all target lesions, where any pathological lymph nodes (whether target or non-target) have a reduction in short axis to less than 10 mm. In some instances, a subject may achieve an objective response (OR), where achieving an OR will generally refer to achieving either a PR or a CR.

In some instances, a subject with a disease, such as a cancer or a tumor, may experience immunotherapy-induced regression of the disease, i.e., immunotherapy-induced regression of the cancer or tumor. Disease regression generally refers to a decrease in severity of the disease or symptoms of the disease, including when the disease does not become completely absent. In cancer, regression generally refers to a decrease in the size of a tumor and/or the extent of cancer in the body of a subject. Disease regression in a subject with a cancer or tumor may manifest as a reduction in the number of tumors, a reduction in the size of one or more tumors, or a combination thereof. Accordingly, cancer or tumor progression and regression may be measured by a variety of means, including but not limited to radiologic imaging, such as e.g., CT and MRI scans. Disease progression and regression generally may be measured by a variety of means, including but not limited to, through radiologic imaging, clinical biomarkers, biopsy (e.g., needle and liquid biopsies), combinations thereof, and the like. Immunotherapy-induced regression represents disease regression that is due to the administration of one or more immunotherapies to the subject, such as e.g., the combination immunotherapy comprising an anti-PD-1 antibody composition and an DR IL-18 composition as described herein. With regard to a cancer or a tumor, immunotherapy-induced regression therefore refers to a reduction in the number of tumors, a reduction in the size of one or more tumors, or a combination thereof, that is due to the administration of one or more immunotherapies to the subject, such as e.g., the combination immunotherapy comprising an anti-PD-1 antibody composition and an DR IL-18 composition as described herein.

Improvements in the condition of the subject may be expressed as improvements in individual subjects or as improvements over one or more cohorts of multiple subjects. For example, a single subject may achieve stable disease, PR, or CR; or all (i.e., 100%) or some number or percentage (e.g., at least 10%, 25%, 50%, 75%, etc.) of subjects of a cohort may achieve stable disease, PR, CR, or a combination thereof. In some instances, at least 10%, 20%, 30%, 40%, 50%, 60%, 70%, or 80%, or 90% of a cohort will achieve stable disease lasting at least 12 months. In some instances, at least 1, 2, 3, 4, 5, 6, 7, 8, 9, or 10 patients of a cohort will achieve an OR. In some instances, at least 1, 2, 3, 4, 5, 6, 7, 8, 9, or 10 patients of a cohort will achieve a PR. In some instances, at least 1, 2, 3, 4, 5, 6, 7, 8, 9, or 10 patients of a cohort will achieve a CR. In some instances, a cohort will display multiple improvements in different categories, including e.g., at least 10%, 20%, 30%, 40%, 50%, 60%, 70%, or 80%, or 90% of the cohort achieves stable disease and at least 1, 2, 3, 4, 5, 6, 7, 8, 9, or 10 patients of the cohort achieve an OR.

In some embodiments, administering successive doses of the anti-PD-1 antibody composition and the DR IL-18 composition results in a reduction in tumor size in the subject. In some embodiments, administering successive doses of the anti-PD-1 antibody composition and the DR IL-18 composition results in a reduction in tumor number in the subject. In some embodiments, administering successive doses of the anti-PD-1 antibody composition and the DR IL-18 results in a reduction in tumor size and a reduction in tumor number in the subject. Decoy-resistant IL-18 polypeptides bind to, and signal through formation of, the IL-18Rα (IL-18 receptor α) and IL-18Rβ (IL-18 receptor β) receptor complex. Decoy-resistant IL-18 polypeptides do not bind to IL-18 binding protein (IL-18BP) or display substantially reduced binding to IL-18BP, such as substantially reduced binding to IL-18BP relative to wild-type (WT) IL-18 (SEQ ID NO: 36) (i.e., as compared to the binding of IL-18BP to WT IL-18 (SEQ ID NO: 36)). In some embodiments, the decoy-resistant IL-18 polypeptide binds to IL-18Rα and does not bind to IL-18BP. In some embodiments, the decoy-resistant IL-18 polypeptide binds to IL-18Rα and has reduced binding to IL-18BP relative to WT IL-18. In some embodiments, the decoy-resistant IL-18 polypeptide is a monomer. In some embodiments, the monomer is not in a protein complex. In some embodiments, the monomer is functional as a single polypeptide. In some embodiments, the decoy-resistant IL-18 polypeptide is not glycosylated. In some embodiments, the decoy-resistant IL-18 polypeptide is glycosylated. In some embodiments, the decoy-resistant IL-18 polypeptide is partially glycosylated. In some embodiments, the decoy-resistant IL-18 polypeptide is at least about 50% glycosylated. In some embodiments, the decoy-resistant IL-18 polypeptide is at most about 50% glycosylated.

In some embodiments, the decoy-resistant IL-18 polypeptide occupies between 10% to about 50% of the IL-18 receptor. In some embodiments, the decoy-resistant IL-18 polypeptide occupies at least about 10%, at least about 15%, at least about 20%, at least about 25%, at least about 30%, at least about 35%, at least about 40%, at least about 45%, at least about 50%, or more of the IL-18 receptor. In some embodiments, the decoy-resistant IL-18 polypeptide occupies at most about 50%, at most about 45%, at most about 40%, at most about 35%, at most about 30%, at most about 25%, at most about 20%, at most about 15%, at most about 10%, or less of the IL-18 receptor. In some embodiments, the decoy-resistant IL-18 polypeptide occupies about 10%, about 15%, about 20%, about 25%, about 30%, about 35%, about 40%, about 45%, or about 50% of the IL-18 receptor.

In some embodiments, administering successive doses of the anti-PD-1 antibody composition and the DR IL-18 composition results in a reduction in tumor size in a tumor resistant to PD-1 checkpoint inhibitors or anti-PD-1 inhibitors. In some embodiments, administering successive doses of the anti-PD-1 antibody composition and the DR IL-18 composition results in a reduction in tumor number in the subject. In some embodiments, administering successive doses of the anti-PD-1 antibody composition and the DR IL-18 composition results in a reduction in tumor size and a reduction in tumor number in the subject.

### Pembrolizumab Composition

The anti-PD-1 antibody composition and the DR IL-18 composition can be administered to the subject beginning on a first day. In some embodiments, a first dose of the successive doses of the anti-PD-1 antibody composition is administered to the subject at least about 60 minutes before a first dose of the successive doses of the DR IL-18 composition is administered to the subject. In some embodiments, the first dose of the successive doses of the anti-PD-1 antibody composition comprises 200 mg pembrolizumab.

In some embodiments, each dose of the successive doses of the anti-PD-1 antibody composition is administered to the subject Q3W. In some embodiments, each dose of the successive doses of the anti-PD-1 antibody composition is administered to the subject about every 21 days.

In some embodiments, each dose of the successive doses of the anti-PD-1 antibody composition comprises a fixed dose of 200 mg pembrolizumab. In some embodiments, each dose of the successive doses of the anti-PD-1 antibody composition comprises about 2 mg/kg pembrolizumab per body weight of the subject (i.e., 2 mg of pembrolizumab per kg body weight of the subject).

In some embodiments, each dose of the successive doses of the anti-PD-1 antibody composition is administered to the subject Q3W as a fixed dose of 200 mg pembrolizumab. In some embodiments, each dose of the successive doses of the anti-PD-1 antibody composition is administered to the subject about every 21 days as a fixed dose of 200 mg pembrolizumab. In some embodiments, each dose of the successive doses of the anti-PD-1 antibody composition is administered to the subject Q3W as a dose of about 2 mg/kg pembrolizumab per body weight of the subject. In some embodiments, each dose of the successive doses of the anti-PD-1 antibody composition is administered to the subject about every 21 days a dose of about 2 mg/kg pembrolizumab per body weight of the subject.

### DR IL-18 Composition

The anti-PD-1 antibody composition and the DR IL-18 composition can be administered to the subject beginning on a first day. In some embodiments, a dose of the DR IL-18 composition is at least one of an initial dose, a first dose, or a lowest dose administered to the subject.

In some embodiments, a first dose of the successive doses of the DR IL-18 composition is administered to the subject at least about 60 minutes after a first dose of the successive doses of the anti-PD-1 antibody composition is administered to the subject. In some embodiments, the first dose of the successive doses of the DR IL-18 composition comprises about 15 μg/kg of the polypeptide per body weight of the subject (i.e., 15 μg of the polypeptide per kg body weight of the subject). In some embodiments, the first dose of the successive doses of the DR IL-18 composition comprises about 20 μg/kg of the polypeptide per body weight of the subject. In some embodiments, the first dose of the successive doses of the DR IL-18 composition comprises about 30 μg/kg of the polypeptide per body weight of the subject. In some embodiments, the first dose of the successive doses of the DR IL-18 composition comprises between about 15 μg/kg to about 30 μg/kg of the polypeptide per body weight of the subject.

In some embodiments, each dose of the successive doses of the DR IL-18 composition is administered to the subject weekly. In some embodiments, each dose of the successive doses of the DR IL-18 composition is administered to the subject about every 7 days. In some embodiments, a next dose of the DR IL-18 composition is administered to the subject at least 6 days after a previous dose of the DR IL-18 composition. In some embodiments, a next dose of the DR IL-18 composition is administered to the subject at most 9 days after a previous dose of the DR IL-18 composition.

In some embodiments, the polypeptide is at a concentration of about 30 mg/mL.

In some embodiments, a dose of the DR IL-18 composition comprises at least about 15 μg/kg of the polypeptide per body weight of the subject. In some embodiments, a dose of the DR IL-18 composition comprises at least about 20 μg/kg of the polypeptide per body weight of the subject. In some embodiments, a dose of the DR IL-18 composition comprises at least about 30 μg/kg of the polypeptide per body weight of the subject. In some embodiments, a dose of the DR IL-18 composition comprises at least about 90 μg/kg of the polypeptide per body weight of the subject. In some embodiments, a dose of the DR IL-18 composition comprises at least about 180 μg/kg of the polypeptide per body weight of the subject. In some embodiments, a dose of the DR IL-18 composition comprises at least about 360 μg/kg of the polypeptide per body weight of the subject. In some embodiments, a dose of the DR IL-18 composition comprises at least about 600 μg/kg of the polypeptide per body weight of the subject. In some embodiments, a dose of the DR IL-18 composition comprises at least about 900 μg/kg of the polypeptide per body weight of the subject. In some embodiments, a dose of the DR IL-18 composition comprises at least about 1200 μg/kg of the polypeptide per body weight of the subject.

In some embodiments, each dose of the DR IL-18 composition comprises between about 15 μg/kg to about 1200 μg/kg of the polypeptide per body weight of the subject. In some embodiments, a dose of the DR IL-18 composition comprises between about 15 μg/kg to about 30 μg/kg, about 30 μg/kg to about 90 μg/kg, about 90 μg/kg to about 180 μg/kg, about 180 μg/kg to about 360 μg/kg, about 360 μg/kg to about 600 μg/kg, about 600 μg/kg to about 900 μg/kg, or about 900 μg/kg to about 1200 μg/kg of the polypeptide per body weight of the subject. In some embodiments, a dose of the DR IL-18 composition comprises about 15 μg/kg, about 30 μg/kg, about 90 μg/kg, about 180 μg/kg, about 360 μg/kg, about 600 μg/kg, about 900 μg/kg, or about 1200 μg/kg of the polypeptide per body weight of the subject.

### DR IL-18 Dose Escalation

Subjects can exhibit a tolerance for a dose of the DR IL-18 composition comprising the polypeptide and can be administered a higher dose of the polypeptide in a subsequent dose of the successive doses of the DR IL-18 composition.

In some embodiments, a subsequent dose of the DR IL-18 composition comprises a greater amount of the polypeptide per body weight of the subject than an amount of the polypeptide administered previously to the subject. In some embodiments, the previous amount of the polypeptide was tolerated by the subject.

In some embodiments, a subsequent dose of the DR IL-18 composition comprises a greater amount of the polypeptide per body weight of the subject than an amount of the polypeptide in a previous dose of the DR IL-18 composition. In some embodiments, the previous dose was tolerated by the subject.

In some embodiments, a subsequent dose of the DR IL-18 composition comprises a same or similar amount of the polypeptide per body weight of the subject to an amount of the polypeptide administered previously to the subject. In some embodiments, the amount of the polypeptide is associated with a recorded Treatment-Emergent Adverse Event in the subject.

In some embodiments, the recorded Treatment-Emergent Adverse Event is a Grade 1 or Grade 2 Treatment-Emergent Adverse Event according to NCI CTCAE Version 5.0. In some embodiments, the recorded Treatment-Emergent Adverse Event is associated with no limitation to mild limitation in activity of the subject. In some embodiments, the recorded Treatment-Emergent Adverse Event is associated with providing no or minimal medical intervention, assistance, or therapy to the subject. In some embodiments, the amount is a highest tolerable amount.

In some embodiments, a subsequent dose of the DR IL-18 composition comprises a same or similar amount of the polypeptide per body weight of the subject to an amount of the polypeptide in a previous dose of the DR IL-18 composition. In some embodiments, the previous dose is associated with causing a recorded Treatment-Emergent Adverse Event in the subject. In some embodiments, the recorded Treatment-Emergent Adverse Event is a Grade 1 or Grade 2 Treatment-Emergent Adverse Event according to NCI CTCAE Version 5.0. In some embodiments, the recorded Treatment-Emergent Adverse Event is as-sociated with no limitation to mild limitation in activity of the subject. In some embodiments, the recorded Treatment-Emergent Adverse Event is associated with providing no or minimal medical intervention, assistance, or therapy to the subject. In some embodiments, the previous dose is a highest tolerable dose.

In some embodiments, a subsequent dose of the DR IL-18 composition comprises a lesser amount of the polypeptide per body weight of the subject than an amount of the polypeptide administered previously to the subject. In some embodiments, the subject did not tolerate the amount of the polypeptide administered previously. In some embodiments, the amount of the polypeptide administered previously is as-sociated with dose limiting toxicity (DLT). In some embodiments, the subsequent dose is a previously tolerated dose.

In some embodiments, a subsequent dose of the DR IL-18 composition comprises a lesser amount of the polypeptide per body weight of the subject than an amount of the polypeptide in a previous dose of the DR IL-18 composition. In some embodiments, the previous dose was not tolerated by the subject. In some embodiments, the previous dose is associated with dose limiting toxicity (DLT). In some embodiments, the subsequent dose is a previously tolerated dose.

### Cycles of Pembrolizumab and DR IL-18 Administration

In some embodiments, the anti-PD-1 and DR IL-18 compositions are administered to the subject is a series of cycles, wherein each cycle comprises about 21 days.

In some embodiments, the anti-PD-1 antibody composition is administered once per cycle. In some embodiments, the DR IL-18 composition is administered one, two, or three times during each cycle.

In some embodiments, the series of cycles comprises a current cycle and a next cycle, wherein the next cycle runs consecutively to the current cycle. In some embodiments, the series of cycles comprises a first cycle, and wherein the first cycle comprises a first day. In some embodiments, a dose of the anti-PD-1 antibody composition comprising 200 mg of pembrolizumab is administered to the subject on the first day of the first cycle. In some embodiments, a dose of the DR IL-18 composition comprising 15 μg/kg of the polypeptide is administered to the subject on the first day of the first cycle. In some embodiments, a dose of the DR IL-18 composition comprising 20 μg/kg of the polypeptide is administered to the subject on the first day of the first cycle. In some embodiments, a dose of the DR IL-18 composition comprising 30 μg/kg of the polypeptide is administered to the subject on the first day of the first cycle. In some embodiments, a dose of the DR IL-18 composition comprising between 15 μg/kg to 30 μg/kg of the polypeptide is administered to the subject on the first day of the first cycle.

In some embodiments, the series of cycles comprises at least 8 cycles. In some embodiments, the series of cycles comprises no more than 35 cycles. In some embodiments, the anti-PD-1 and DR IL-18 compositions are administered until the subject exhibits a complete response (CR) and has been treated for at least 8 cycles. In some embodiments, a complete response is measured by integrated assessments of the magnitude and extent of changes in tumor dimension that conveniently categorize and describe treatment effects.

### Discontinuation

In some embodiments, the anti-PD-1 and DR IL-18 compositions are administered for at least 24 weeks. In some embodiments, the anti-PD-1 and DR IL-18 compositions are administered until the subject has been administered at least 8 doses of the anti-PD-1 antibody composition. In some embodiments, the anti-PD-1 and DR IL-18 compositions are administered until the subject exhibits a complete response (CR) and has been treated for at least 24 weeks or has been administered at least 8 doses of the anti-PD-1 antibody composition. In some embodiments, the anti-PD-1 and DR IL-18 compositions are administered until the subject exhibits a complete response (CR) and has been treated for at least 8 cycles. In some embodiments, the anti-PD-1 and DR IL-18 compositions are administered until the subject exhibits a complete response (CR) and has been administered at least 8 doses of the anti-PD-1 antibody composition. In some embodiments, the anti-PD-1 and DR IL-18 compositions are administered until the subject has been administered at least about 24 doses of the anti-PD-1 antibody composition.

### Withdrawal

In some embodiments, the anti-PD-1 and DR IL-18 compositions are administered until the subject exhibits an intervention-related toxicity specified as a reason for permanent discontinuation. In some embodiments, the intervention-related toxicity is at least one of a Grade 4, Grade 3, or recurrent Grade 2 Treatment-Emergent Adverse Event according to NCI CTCAE Version 5.0. In some embodiments, the Treatment-Emergent Adverse Event is associated with marked or severe limitation to extreme limitation in activity of the subject. In some embodiments, the Treatment-Emergent Adverse Event is life threatening or requires significant medical intervention or hospitalization. In some embodiments, the anti-PD-1 and DR IL-18 compositions are administered for at most about 2 years. In some embodiments, at most 35 doses of the anti-PD-1 antibody composition are administered to the subject.

### Order of Administration

The anti-PD-1 antibody composition and the DR IL-18 composition can be administered to the subject on the same day (e.g., the first day). In some embodiments, a dose of the anti-PD-1 antibody composition is administered to the subject at least about 60 minutes before a dose of the DR IL-18 composition is administered to the subject. In some embodiments, a dose of the DR IL-18 composition is administered to the subject at least about 60 minutes after a dose of the anti-PD-1 antibody composition is administered to the subject. In some embodiments, (a) and (b) are performed sequentially. In some embodiments, (a) is performed before (b). In some embodiments, (b) is performed before (a). In some embodiments, (a) and (b) are performed simultaneously. In some embodiments, (a) and (b) are performed concurrently.

### Routes of Administration

In some embodiments, each dose of the anti-PD-1 antibody composition is administered to the subject by intravenous infusion. In some embodiments, wherein a dose of the anti-PD-1 antibody composition is administered to the subject by intravenous infusion. In some embodiments, wherein a dose of the anti-PD-1 antibody composition is administered to the subject by subcutaneous injection. In some embodiments, wherein each dose of the DR IL-18 composition is administered to the subject by subcutaneous injection. In some embodiments, wherein a dose of the DR IL-18 composition is administered to the subject by subcutaneous injection.

### Indications of the Subject

One aspect of the present disclosure provides methods for treating a disease in a subject in need thereof. In some embodiments, the disease is cancer. In some embodiments, the cancer is a solid tumor.

In some embodiments, the cancer is leukemia, lymphoma, melanoma, neuroendocrine tumor, carcinoma and/or sarcoma. Non-limiting examples of cancers include lymphoma, sarcoma, bladder cancer, biliary tract cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional non-limiting examples of cancer include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, and prostate cancer.

In some embodiments, the cancer is squamous cell carcinoma. In some embodiments, the cancer is prostate cancer. In some embodiments, the cancer is colorectal cancer.

In some embodiments, the cancer is leukemia. Leukemia can be progressive, malignant diseases of the blood-forming organs and can have distorted proliferation and development of leukocytes and their precursors in the blood and bone marrow. Leukemia can be clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Non-limiting examples of leukemias include acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, 44asophilic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

In some embodiments, the cancer is sarcoma. Sarcoma can be a tumor which can be made up of a substance like the embryonic connective tissue and can be composed of closely packed cells embedded in a fibrillar and/or homogeneous substance. Non-limiting examples of sarcomas include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewings sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocyte sarcoma, Rous sarcoma, serocystic sarcoma, synovial sarcoma, or telangiectaltic sarcoma.

In some embodiments, the cancer is melanoma. Melanoma can be a tumor arising from the melanocytic system of the skin and other organs. Non-limiting examples of melanomas include acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

In some embodiments, the cancer is a solid tumor. Non-limiting examples of solid tumor cancers include bladder cancer, breast cancer, cervical cancer, colon cancer, rectal cancer, endometrial cancer, kidney cancer, lip cancer, oral cancer, liver cancer, melanoma, mesothelioma, non-small cell lung cancer (NSCLC), nonmelanoma skin cancer, ovarian cancer, pancreatic cancer, prostate cancer, sarcoma, small cell lung cancer, and thyroid cancer.

In some embodiments, the solid tumor is selected from the group consisting of melanoma, Merkel cell carcinoma, renal cell carcinoma, urothelial, non-small cell lung cancer (NSCLC), triple-negative breast cancer (TNBC), squamous cell carcinoma of head and neck (SCCHN), microsatellite instability high (MSI-H) tumors, high tumor mutation burden (TMB-H) tumors, mismatch repair deficient tumors, gastric, cervical, endometrial, cutaneous squamous, small cell lung, esophageal, hepatocellular carcinoma (HCC), platinum-resistant ovarian cancer, and any combination thereof.

In some embodiments, the solid tumor is melanoma, renal cell carcinoma, triple-negative breast cancer, NSCLC, SCCHN, or MSI-H tumors. In some embodiments, the solid tumor is melanoma. In some embodiments, the solid tumor is renal cell carcinoma. In some embodiments, the solid tumor is TNBC. In some embodiments, the solid tumor is NSCLC. In some embodiments, the solid tumor is SCCHN. In some embodiments, the solid tumor is MSI-H tumors.

In some embodiments, cancer or solid tumor is associated with melanoma, non-small cell lung cancer (NSCLC), small cell lung cancer, head and neck squamous cell cancer (HNSCC), classical Hodgkin Lymphoma (cHL), primary mediastinal large B-cell lymphoma (PMBCL), urothelial carcinoma, microsatellite instability-high (MSI-H) or mismatch repair deficient cancer (dMMR) cancer, microsatellite instability-high or mismatch repair deficient colorectal cancer (CRC), colorectal cancer, gastric cancer, esophageal or gastroesophageal junction cancer, esophageal or gastroesophageal junction adenocarcinoma, locally advanced or metastatic esophageal or gastroesophageal junction (GEJ) carcinoma, malignant pleural mesothelioma, cervical cancer, hepatocellular carcinoma (HCC), Merkel cell carcinoma (MCC), kidney cancer, renal cell carcinoma (RCC), bladder cancer, endometrial carcinoma, liver cancer, tumor mutational burden-high (TMB-H) cancer, cutaneous squamous cell carcinoma (cSCC), triple-negative breast cancer (TNBC), or any combination thereof.

In some embodiments, the cancer or solid tumor is associated with melanoma, non-small cell lung cancer (NSCLC), small cell lung cancer, head and neck squamous cell cancer (HNSCC), classical Hodgkin Lymphoma (cHL), primary mediastinal large B-cell lymphoma (PMBCL), urothelial carcinoma, microsatellite instability-high (MSI-H) or mismatch repair deficient cancer (dMMR) cancer, microsatellite instability-high or mismatch repair deficient colorectal cancer (CRC), colorectal cancer, gastric cancer, esophageal or gastroesophageal junction cancer, esophageal or gastroesophageal junction adenocarcinoma, locally advanced or metastatic esophageal or gastroesophageal junction (GEJ) carcinoma, malignant pleural mesothelioma, cervical cancer, ovarian cancer, hepatocellular carcinoma (HCC), Merkel cell carcinoma (MCC), kidney cancer, renal cell carcinoma (RCC), bladder cancer, endometrial carcinoma, liver cancer, tumor mutational burden-high (TMB-H) cancer, cutaneous squamous cell carcinoma (cSCC), triple-negative breast cancer (TNBC), or any combination thereof. In some embodiments, the cancer or solid tumor is resistant to PD-1 checkpoint inhibitors.

In some embodiments, the cancer or solid tumor is associated with Melanoma, Merkel cell, RCC, urothelial, NSCLC (with no epidermal growth factor receptor, TRK receptor, or anaplastic lymphoma kinase positive mutations/fusions), TNBC, SCCHN, MSI-H, TMB-H or mismatch repair deficient, gastric, cervical, endometrial, cutaneous squamous, small cell lung, esophageal, HCC, or any combination thereof, and wherein the cancer or solid tumor is resistant to PD-1 checkpoint inhibitors.

In some embodiments, the cancer or solid tumor is associated with platinum-resistant ovarian cancer or microsatellite stable colorectal cancer.

In some embodiments, the methods of the present disclosure are useful for treating a tumor or cancer that is resistant to immune checkpoint inhibitors (ICIs). Examples of immune checkpoint inhibitors include, but are not limited to: anti-PD1 agents such as an anti-PD1 antibody (e.g., zimberelimab, nivolumab, cemiplimab, dostarlimab), anti-PD-L1 agents such as an anti-PD-L1 antibody (e.g., avelumab, durvalumab, atezolizumab), anti-PD-L2 agents such as an anti-PD-L2 antibody, anti-CTLA4 (e.g., ipilimumab, tremelimumab), anti-TIM3, anti-TIGIT, anti-LAG3 (e.g., relatlimab), anti-B7H3 (e.g., enoblituzumab), anti-B7H4, anti-VISTA, anti-BTLA, anti-CD47, anti-SIRP alpha, anti-CD48, anti-CD155, anti-CD160, anti-TREM2, anti-IDO1, anti-Adenosine 2A receptor, anti-Aryl hydrocarbon receptor, anti-KIR, and anti-LILRB2. Examples of targets of immune checkpoint inhibitors include but are not limited to: PD-L1, PD1, CTLA4, TIM3, TIGIT, LAG3, B7H3, B7H4, VISTA, BTLA, CD47, SIRP alpha, CD48, CD155, CD160, TREM2, IDO1, Adenosine 2A receptor, Aryl hydrocarbon receptor, KIR, and LILRB2, or any combination thereof.

In some embodiments, the disease is cancer. Cancer can be a hyperproliferation of cells which can have unregulated growth, lack of differentiation, local tissue invasion, and/or metastasis. In some embodiments, the cancer is a solid tumor. A tumor can be an abnormal growth of cells or tissues, (e.g., of malignant type or benign type).

### CRS Prophylaxis Administration

Cytokine release syndrome (CRS) may occur in a subject following dosing with a DR IL-18 composition. In some embodiments, the method further comprises administering to the subject an additional treatment. In some embodiments, the additional treatment is a CRS prophylactic agent. Consistent with the definitions provided above, articles “a” and “an” refer to one or to more than one (i.e., to at least one) of the grammatical object of the article and thus, reference herein to administration of a CRS prophylactic agent or a dose will refer to administration of one or to more than one CRS prophylactic agents (i.e., one or more CRS prophylactic agents), to administration of one or to more than one dose (i.e., one or more doses), to administration of at least one CRS prophylactic agent, or to administration of at least one dose. Administration of one or more CRS prophylactic agents may be referred to herein as CRS prophylaxis.

CRS prophylaxis, or a CRS prophylactic agent, can be administered to a subject starting from the first dose of the DR IL-18 composition. In some embodiments, the CRS prophylactic agent is administered as premedication or post-dose medication to a dose of the DR IL-18 composition. In some embodiments, the method further comprises administering a dose of a CRS prophylactic agent to the subject with or before a dose of the successive doses of the DR IL-18 composition. In some embodiments, the method comprises administering a dose of a CRS prophylactic agent to the subject with or before each dose of the successive doses of the DR IL-18 composition.

One aspect of the present disclosure provides methods for treating a disease in a subject in need thereof. In some embodiments, the disease is cancer. In some embodiments, the cancer is a solid tumor. One aspect of the present disclosure provides methods of treating the disease in the subject in need thereof comprising (a) administering successive doses of an anti-PD-1 antibody composition comprising pembrolizumab to the subject; (b) administering successive doses of a decoy-resistant (DR) IL-18 composition comprising a polypeptide to the subject, wherein the polypeptide is a modified IL-18 polypeptide comprising an amino acid sequence set forth in any one of SEQ ID NOs: 36-43; and (c) administering a dose of a cytokine release syndrome (CRS) prophylactic agent to the subject. One aspect of the present disclosure provides methods of treating the disease in the subject in need thereof comprising (a) administering successive doses of an anti-PD-1 antibody composition comprising pembrolizumab to the subject; (b) administering successive doses of a decoy-resistant (DR) IL-18 composition comprising a polypeptide to the subject, wherein the polypeptide is a modified IL-18 polypeptide comprising an amino acid sequence set forth in any one of SEQ ID NOs: 36-43; and (c) administering a dose of a cytokine release syndrome (CRS) prophylactic agent to the subject; thereby causing immunotherapy-induced regression of the disease in the subject. In some embodiments, the dose of the CRS prophylactic agent is administered with or before a dose of the successive doses of the DR IL-18 composition. In some embodiments, the method comprises administering a dose of a CRS prophylactic agent to the subject with or before each dose of the successive doses of the DR IL-18 composition. In some embodiments, the dose of the CRS prophylactic agent comprises at least one of a NSAID, acetaminophen, diphenhydramine, histamine H1 antagonist, famotidine, H2 blocker, or fluid administered to the subject. In some embodiments, the dose of the CRS prophylactic agent is administered to the subject orally or intravenously.

In some embodiments, the CRS prophylactic agent is administered orally. In some embodiments, one or more CRS prophylactic agents of CRS prophylaxis are administered orally. In some embodiments, the CRS prophylactic agent is administered subcutaneously. In some embodiments, one or more CRS prophylactic agents of CRS prophylaxis are administered subcutaneously. In some embodiments, the CRS prophylactic agent is administered intramuscularly. In some embodiments, one or more CRS prophylactic agents of CRS prophylaxis are administered intramuscularly. In some embodiments, the CRS prophylactic agent is administered intravenously. In some embodiments, one or more CRS prophylactic agents of CRS prophylaxis are administered intravenously. In some embodiments, the CRS prophylactic agent is administered intrathecally. In some embodiments, the CRS prophylactic agent is administered rectally. In some embodiments, the CRS prophylactic agent is administered vaginally. In some embodiments, the CRS prophylactic agent is administered nasally. In some instances, multiple routes of administration are employed, including e.g., where one or more CRS prophylactic agents of a CRS prophylaxis regimen are administered orally and one or more CRS prophylactic agents of the CRS prophylaxis regimen are administered intravenously.

In some embodiments, the CRS prophylactic agent is administered hourly. In some embodiments, the CRS prophylactic agent is administered about every several hours. In some embodiments, the CRS prophylactic agent is administered twice daily. In some embodiments, the CRS prophylactic agent is administered daily. In some embodiments, the CRS prophylactic agent is administered every six days. In some embodiments, the CRS prophylactic agent is administered twice a week. In some embodiments, the CRS prophylactic agent is administered weekly. In some embodiments, the CRS prophylactic agent is administered every two weeks. In some embodiments, the CRS prophylactic agent is administered every three weeks. In some embodiments, the CRS prophylactic agent is administered monthly. In some embodiments, the CRS prophylactic agent is administered every two months. In some embodiments, the CRS prophylactic agent is administered every three months. In some embodiments, the CRS prophylactic agent is administered every four months. In some embodiments, the CRS prophylactic agent is administered every six months. In some embodiments, the CRS prophylactic agent is administered once a year.

In some embodiments, the CRS prophylactic agent reduces the display of the symptoms of cytokine release syndrome (CRS). In some embodiments, the administering the CRS prophylactic agent reduces the display of the symptoms of CRS as compared to an equivalent subject who did not receive the CRS prophylactic agent. In some embodiments, the administering the CRS prophylactic agent reduces the display of the symptoms of CRS as compared to a corresponding subject having received an equivalent therapy but who did not receive the CRS prophylactic agent. In some embodiments, administering to the subject the CRS prophylactic agent results in a reduced display of symptoms of CRS in the subject as compared to an equivalent subject administered about 30 mg/ml or more of the DR IL-18 composition. In some embodiments, administering to the subject the CRS prophylactic agent results in a reduced display of symptoms of CRS in the subject as compared to a corresponding subject having received an equivalent therapy that includes administration of about 30 mg/ml or more of the DR IL-18 composition. In some embodiments, administering to the subject the CRS prophylactic agent results in a reduced display of symptoms of CRS in the subject as compared to an equivalent subject administered about 30 μg/kg or more of the DR IL-18 composition. In some embodiments, administering to the subject the CRS prophylactic agent results in a reduced display of symptoms of CRS in the subject as compared to a corresponding subject having received an equivalent therapy that includes administration of about 30 μg/kg or more of the DR IL-18 composition.

In some embodiments, CRS prophylaxis reduces the display of the symptoms of CRS in a subject administered a combination therapy comprising a DR IL-18 composition, including where the display of symptoms are reduced relative to a subject administered the same therapy without CRS prophylaxis. In some embodiments, CRS prophylaxis reduces the display of the symptoms of CRS in a subject administered a combination therapy comprising administration of a DR IL-18 composition comprising at least about 30 μg/kg or more of a DR-IL-18 polypeptide, including where the display of symptoms are reduced relative to a subject administered the same therapy without CRS prophylaxis. In some embodiments, CRS prophylaxis reduces the display of the symptoms of CRS in a subject administered an anti-PD-1 antibody composition and DR IL-18 composition combination therapy, including where the display of symptoms are reduced relative to a subject administered the same therapy without CRS prophylaxis.

In some embodiments, administration of CRS prophylaxis will prevent the onset or progression of CRS in a subject administered a combination therapy that comprises administration of a DR IL-18 composition comprising a DR-IL-18 polypeptide, including e.g., at least about 30 μg/kg or more of a DR-IL-18 polypeptide. For example, administration of CRS prophylaxis will prevent the onset of or progression to grade 5 CRS, grade 4 CRS or higher, and/or grade 3 CRS or higher, including e.g., where CRS grading is performed according to Consensus American Society for Transplantation and Cellular Therapy (ASTCT) grading for CRS as described in Lee et al. Biology of Blood and Marrow Transplantation. 25 (2019) 625-639. In some instances, a lack of onset or progression of CRS may be indicated by a lack of fever (temp. greater than or equal to 38 deg. C.), a lack of hypotension, hypotension that does not require vasopressors, hypotension requiring only one vasopressor with or without vasopressin, a lack of hypoxia, hypoxia requiring only low-flow nasal canula or blow-by, hypoxia requiring high-flow nasal-canula (or facemask, nonrebreather mask, or venturi mask) but not requiring positive pressure, or a combination thereof.

In some embodiments, CRS prophylaxis in a subject administered a combination therapy that comprises administration of a DR IL-18 composition comprising a DR-IL-18 polypeptide, including e.g., at least about 30 μg/kg or more of a DR-IL-18 polypeptide, will negate the need for later treating the subject for CRS. For example, in some instances, CRS prophylaxis in such scenarios will mean that administration of immunosuppressants, such as, e.g., corticosteroids (e.g., glucocorticoids), anti-interleukin 6 (IL-6) agents (e.g., tocilizumab, siltuximab, clazakizumab), TNF-α signaling inhibitors (e.g., etanercept), T cell-depleting antibodies (e.g., alemtuzumab), IL-1R-based inhibitors (e.g., anakinra), cyclophosphamide, Bruton's tyrosine kinase (BTK) inhibitors (e.g., ibrutinib), combinations thereof, and the like will not be necessary and will not be administered. In some embodiments, a subject receiving CRS prophylaxis and administered a combination therapy that comprises administration of a DR IL-18 composition comprising a DR-IL-18 polypeptide, including e.g., at least about 30 μg/kg or more of a DR-IL-18 polypeptide, will not require and will not be administered an immunosuppressant to treat CRS. For example, in some instances, such a subject will not be administered any corticosteroids (e.g., glucocorticoids) and/or any anti-interleukin 6 (IL-6) agents during a cycle of the combination therapy or during the entire course of the combination therapy.

In some embodiments, the CRS prophylactic agent is at least one of a non-steroidal anti-inflammatory drug (NSAID), acetaminophen, diphenhydramine, histamine H1 antagonist, famotidine, H2 blocker, or fluid.

### NSAID

In some embodiments, the CRS prophylactic agent is a non-steroidal anti-inflammatory drug (NSAID). In some embodiments, the NSAID is administered to the subject between about 1 hour to 2 hours prior to administering a dose of the DR IL-18 composition. In some embodiments, the NSAID is administered to the subject for at least about 48 hours after administering the dose of the DR IL-18 composition. In some embodiments, the NSAID comprises indomethacin or ibuprofen. In some embodiments, the indomethacin is administered to the subject at 25 mg three times daily. In some embodiments, the ibuprofen is administered to the subject at between 200 mg to 600 mg every 6 to 8 hours. In some embodiments, the NSAID is administered to the subject orally.

Non-limiting examples of NSAIDs include ibuprofen, naproxen, diclofenac, diflunisal, fenoprofen, flurbiprofen, ketoprofen, meloxicam, nabumetone, oxaproin, piroxicam, etodolac, indomethacin, ketorolac, nabumetone, sulindac, tolmetin, rofecoxib, valdecoxib, celecoxib, mefenamic acid, etoricoxib, indomethacin, or aspirin. In some embodiments, the NSAID is between about 25 mg to about 600 mg. In some embodiments, the NSAID is between about 25 mg to about 50 mg, between about 50 mg to about 75 mg, between about 75 mg to about 100 mg, between about 100 mg to about 125 mg, between about 125 mg to about 150 mg, between about 150 mg to about 175 mg, between about 175 mg to about 200 mg, between about 200 mg to about 225 mg, between about 225 mg to about 250 mg, between about 250 mg to about 275 mg, between about 275 mg to about 300 mg, between about 300 mg to about 325 mg, between about 325 mg to about 350 mg, between about 350 mg to about 375 mg, between about 375 mg to about 400 mg, between about 400 mg to about 425 mg, between about 425 mg to about 450 mg, between about 450 mg to about 475 mg, between about 475 mg to about 500 mg, between about 500 mg to about 525 mg, between about 525 mg to about 550 mg, between about 550 mg to about 575 mg, or between 575 mg to about 600 mg. In some embodiments, the NSAID is between about 200 mg to about 600 mg.

In some embodiments, the NSAID is at least about 25 mg, at least about 50 mg, at least about 75 mg, at least about 100 mg, at least about 125 mg, at least about 150 mg, at least about 175 mg, at least about 200 mg, at least about 225 mg, at least about 250 mg, at least about 275 mg, at least about 300 mg, at least about 325 mg, at least about 350 mg, at least about 375 mg, at least about 400 mg, at least about 425 mg, at least about 450 mg, at least about 475 mg, at least about 500 mg, at least about 525 mg, at least about 550 mg, at least about 575 mg, at least about 600 mg, or more.

In some embodiments, the NSAID is at most about 600 mg, at most about 575 mg, at most about 550 mg, at most about 525 mg, at most about 500 mg, at most about 475 mg, at most about 450 mg, at most about 425 mg, at most about 400 mg, at most about 375 mg, at most about 350 mg, at most about 325 mg, at most about 300 mg, at most about 275 mg, at most about 250 mg, at most about 225 mg, at most about 200 mg, at most about 175 mg, at most about 150 mg, at most about 125 mg, at most about 100 mg, at most about 75 mg, at most about 50 mg, at most about 25 mg, or less.

In some embodiments, the NSAID is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg.

### Acetaminophen

In some embodiments, the CRS prophylactic agent is acetaminophen. In some embodiments, the acetaminophen is administered to the subject at 650 mg every 4 to 6 hours. In some embodiments, the acetaminophen is administered for at least 24 hours after administering a dose of the DR IL-18 composition. In some embodiments, the acetaminophen is administered to the subject at 650 mg every 4 to 6 hours beginning 1 hour prior to administering a dose of the DR IL-18 composition. In some embodiments, the acetaminophen is administered for at least 48 hours after administering a dose of the DR IL-18 composition. In some embodiments, the acetaminophen is administered to the subject orally.

In some embodiments, the acetaminophen is between about 200 mg to about 800 mg. In some embodiments, the acetaminophen is between about 600 mg to about 800 mg.

In some embodiments, the acetaminophen is at least about 200 mg, at least about 250 mg at least about 300 mg, at least about 350 mg, at least about 400 mg, at least about 450 mg, at least about 500 mg, at least about 550 mg, at least about 600 mg, at least about 650 mg, at least about 700 mg, at least about 750 mg, at least about 800 mg, or more. In some embodiments, the acetaminophen is at most about 800 mg, at most about 750 mg, at most about 700 mg, at most about 650 mg, at most about 600 mg, at most about 550 mg, at most about 500 mg, at most about 450 mg, at most about 400 mg, at most about 350 mg, at most about 300 mg, at most about 250 mg, at most about 200 mg, or less. In some embodiments, the acetaminophen is about 200 mg, about 250 mg, about 300 mg, about 350 mg, about 400 mg, about 450 mg, about 500 mg, about 550 mg, about 600 mg, about 650 mg, about 700 mg, about 750 mg, or about 800 mg. In some embodiments, the acetaminophen is about 650 mg.

### Histamine H1 Antagonists

In some embodiments, the CRS prophylactic agent is a histamine H1 antagonist. In some embodiments the histamine H1 antagonist is administered at 50 mg about 30 minutes to 60 minutes prior to administering a dose of the DR IL-18 composition. In some embodiments, the histamine H1 antagonist is administered intravenously. In some embodiments, the histamine H1 antagonist is administered orally.

Non-limiting examples of histamine H1 antagonists include brompheniramine, clemastine, dexchlorpheniramine dimenhydrinate, diphenhydramine, doxylamine, hydroxyzine, phenindamine, azelastine, loratadine, cetirizine, desloratadine, and fexofenadine, mepyramine, chlorpheniramine, promethazine, cyproheptadine. In some embodiments, the histamine H1 antagonist is between about 20 to about 100 mg. In some embodiments, the histamine H1 antagonist is at least about 20 mg, at least about 30 mg, at least about 40 mg, at least about 50 mg, at least about 60 mg, at least about 70 mg, at least about 80 mg, at least about 90 mg, at least about 100 mg, or more. In some embodiments, the histamine H1 antagonist is at most about 100 mg, at most about 90 mg, at most about 80 mg, at most about 70 mg, at most about 60 mg, at most about 50 mg, at most about 40 mg, at most about 30 mg, at most about 20 mg, or less. In some embodiments, the histamine H1 antagonist is about 20 mg, about 30 mg, about 40 mg, about 50 mg, about 60 mg, about 70 mg, about 80 mg, about 90 mg, or about 100 mg. In some embodiments, the histamine H1 antagonist is about 50 mg.

### Diphenhydramine

In some embodiments, the CRS prophylactic agent is diphenhydramine. In some embodiments the diphenhydramine is administered at 50 mg about 30 minutes to 60 minutes prior to administering a dose of the DR IL-18 composition. In some embodiments, the diphenhydramine is administered intravenously. In some embodiments, the diphenhydramine is administered orally.

In some embodiments, the diphenhydramine is between about 20 to about 100 mg. In some embodiments, the diphenhydramine is at least about 20 mg, at least about 30 mg, at least about 40 mg, at least about 50 mg, at least about 60 mg, at least about 70 mg, at least about 80 mg, at least about 90 mg, at least about 100 mg, or more. In some embodiments the diphenhydramine is at most about 100 mg, at most about 90 mg, at most about 80 mg, at most about 70 mg, at most about 60 mg, at most about 50 mg, at most about 40 mg, at most about 30 mg, at most about 20 mg, or less. In some embodiments, the diphenhydramine is about 20 mg, about 30 mg, about 40 mg, about 50 mg, about 60 mg, about 70 mg, about 80 mg, about 90 mg, or about 100 mg. In some embodiments, the diphenhydramine is about 50 mg.

### H2 Blocker

In some embodiments, the CRS prophylactic agent is a histamine H2 antagonist (H2 blocker). In some embodiments, the H2 blocker is administered at 20 mg about 30 minutes to 60 minutes prior to administering a dose of the DR IL-18 composition. In some embodiments, the H2 blocker is administered intravenously. In some embodiments, the H2 blocker is administered at between 20 mg to 40 mg about 30 minutes to 60 minutes prior to administering a dose of the DR IL-18 composition. In some embodiments, the H2 blocker is administered orally.

In some embodiments, the H2 blocker is cimetidine, ranitidine, or nizatidine. In some embodiments, the H2 blocker is cimetidine. In some embodiments, the H2 blocker is ranitidine. In some embodiments, the H2 blocker is nizatidine. In some embodiments, the H2 blocker is between 10 mg to about 60 mg. In some embodiments, the H2 blocker is between about 20 mg to about 40 mg. In some embodiments, the famotidine is at least about 10 mg, at least about 20 mg, at least about 30 mg, at least about 40 mg, at least about 50 mg, at least about 60 mg, or more. In some embodiments, the H2 blocker is at most about 60 mg, at most about 50 mg, at most about 40 mg, at most about 30 mg, at most about 20 mg, at most about 10 mg, or less. In some embodiments, the H2 blocker is about 10 mg, about 20 mg, about 30 mg, about 40 mg, about 50 mg, or about 60 mg.

### Famotidine

In some embodiments, the CRS prophylactic agent is famotidine. In some embodiments, the famotidine is administered at 20 mg about 30 minutes to 60 minutes prior to administering a dose of the DR IL-18 composition. In some embodiments, the famotidine is administered intravenously. In some embodiments, the famotidine is administered at between 20 mg to 40 mg about 30 minutes to 60 minutes prior to administering a dose of the DR IL-18 composition. In some embodiments, the famotidine is administered orally.

In some embodiments, the famotidine is between 10 mg to about 60 mg. In some embodiments, the famotidine is between about 20 mg to about 40 mg. In some embodiments, the famotidine is at least about 10 mg, at least about 20 mg, at least about 30 mg, at least about 40 mg, at least about 50 mg, at least about 60 mg, or more. In some embodiments, the famotidine is at most about 60 mg, at most about 50 mg, at most about 40 mg, at most about 30 mg, at most about 20 mg, at most about 10 mg, or less. In some embodiments, the famotidine is about 10 mg, about 20 mg, about 30 mg, about 40 mg, about 50 mg, or about 60 mg.

### Fluids

In some embodiments, a subject has hypotension or is at risk of having hypotension. In some embodiments, administering the dose of the CRS prophylactic agent comprises administering a fluid to the subject. In some embodiments, the fluid comprises at most 3 liters of fluid (e.g., water, juice, sports drink, or IV fluids) within 24 hours after administering a dose of the DR IL-18 composition. In some embodiments, the fluid is administered intravenously. In some embodiments, the fluid is administered orally.

In some embodiments, the administering the CRS prophylactic agent results in a reduced display of symptoms of cytokine release syndrome (CRS). Non-limiting examples of symptoms of CRS include fever, chills, tiredness, nausea, vomiting, diarrhea, headache, cough, low blood pressure, joint pain, muscle pain, skin rash, shortness of breath, confusion, dizziness, difficulty swallowing, increased heart rate, reduced heart function, irregular heartbeat, organ failure, and swelling. In some embodiments, the administering results in a reduced display of symptoms of cytokine release syndrome as compared to an equivalent subject administered between about 30 mg/ml to about 100 mg/ml of the DR IL-18 composition. In some embodiments, the administering results in a reduced display of symptoms of cytokine release syndrome as compared to a corresponding subject having been administered an equivalent therapy comprising between about 30 mg/ml to about 100 mg/ml of the DR IL-18 composition. In some embodiments, the administering results in a reduced display of symptoms of CRS as compared to an equivalent subject administered between about 30 mg/ml to about 40 mg/ml, between about 40 mg/ml to about 50 mg/ml, between about 50 mg/ml to about 60 mg/ml, between about 60 mg/ml to about 70 mg/ml, between about 70 mg/ml to about 80 mg/ml, between about 80 mg/ml to about 90 mg/ml, or between about 90 mg/ml to about 100 mg/ml of the DR IL-18 composition. In some embodiments, the administering results in a reduced display of symptoms of CRS as compared to a corresponding subject having been administered an equivalent therapy between about 30 mg/ml to about 40 mg/ml, between about 40 mg/ml to about 50 mg/ml, between about 50 mg/ml to about 60 mg/ml, between about 60 mg/ml to about 70 mg/ml, between about 70 mg/ml to about 80 mg/ml, between about 80 mg/ml to about 90 mg/ml, or between about 90 mg/ml to about 100 mg/ml of the DR IL-18 composition.

### CRS Prophylaxis Combinations

Any of the herein described groups of CRS prophylactic agents and individual CRS prophylactic agents and any of the herein described method of using such groups and individual CRS prophylactic agents may be combined into a useful CRS prophylaxis combination.

For example, in some embodiments, CRS prophylaxis administered to a subject will include at least a NSAID and at least a histamine H1 antagonist; at least acetaminophen and at least a histamine H1 antagonist; at least a NSAID and at least acetaminophen; or at least a NSAID, at least a histamine H1 antagonist, and at least acetaminophen. Such NSAID, histamine H1 antagonist, and acetaminophen used in such combinations may be administered according to the administration and/or dosing of each agent as described herein. In some embodiments, CRS prophylaxis administered to a subject will include at least indomethacin or ibuprofen and at least diphenhydramine; at least acetaminophen and at least diphenhydramine; at least indomethacin or ibuprofen and at least acetaminophen; or at least indomethacin or ibuprofen, at least diphenhydramine, and at least acetaminophen. Such indomethacin or ibuprofen, diphenhydramine, and acetaminophen used in such combinations may be administered according to the administration and/or dosing of each agent as described herein. In some embodiments, such combinations will further include an H2 blocker, including e.g., where the H2 blocker used is famotidine and the famotidine is administered according to the administration and/or dosing described herein.

In some embodiments, CRS prophylaxis will include at least an NSAID, a histamine H1 antagonist, and acetaminophen, including e.g., where (i) the NSAID is oral indomethacin administered at 50 mg to 100 mg per day (inc., e.g., at 75 mg per day), or oral ibuprofen administered at 600 mg to 2,400 mg per day (inc. e.g., at 800 mg to 1,800 mg per day); (ii) the histamine H1 antagonist is intravenous or oral diphenhydramine administered at a dose of 25 mg to 100 mg (inc. e.g., a dose of 50 mg); and (iii) the acetaminophen is oral acetaminophen administered at 350 mg to 4,000 mg per day (inc. e.g., at 650 mg to 3,900 mg per day). In some embodiments, such a combination will further include an H2 blocker, including e.g., where the H2 blocker used is famotidine and the famotidine is administered according to the administration and/or dosing described herein.

In some embodiments, a CRS prophylaxis combination described herein will reduce the display of one or more symptoms of CRS, including reducing the one or more symptoms as compared to a subject administered the same treatment protocol without the CRS prophylaxis. In some embodiments, a CRS prophylaxis combination described herein, such as the combination described above, will prevent a subject with cancer, treated with a combination therapy that includes administration of DR IL-18 composition comprising a polypeptide that is a modified IL-18 polypeptide comprising an amino acid sequence set forth in any one of SEQ ID NOs: 36-43 (such as, e.g., SEQ ID NO:41), from experiencing one or more (including all) symptoms of grade 3 or higher CRS or grade 4 CRS. In some embodiments, a CRS prophylaxis combination described herein, such as the combination described above, will prevent a subject with cancer, treated with a combination therapy that includes administration of DR IL-18 composition comprising a polypeptide that is a modified IL-18 polypeptide comprising an amino acid sequence set forth in any one of SEQ ID NOs: 36-43 (such as, e.g., SEQ ID NO:41), from requiring treatment for CRS, including treatment with one or more (or any) immunosuppressants, such as e.g., a corticosteroid, an anti-interleukin 6 (IL-6) agents (such as e.g., tocilizumab), or any combination thereof.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. A method of treating a disease in a subject in need thereof, the method comprising: (a) administering successive doses of an anti-PD-1 antibody composition comprising pembrolizumab to the subject; and (b) administering successive doses of a decoy-resistant (DR) IL-18 composition comprising a polypeptide to the subject, wherein the polypeptide is a modified IL-18 polypeptide comprising an amino acid sequence set forth in any one of SEQ ID NOs: 36-43; thereby causing immunotherapy-induced regression of the disease in the subject.
2. The method of aspect 1, further comprising administering a dose of a cytokine release syndrome (CRS) prophylactic agent to the subject.
3. The method of aspect 2, wherein the dose of the CRS prophylactic agent is administered with or before a dose of the successive doses of the DR IL-18 composition.
4. The method of aspect 2 or 3, comprising administering a dose of the CRS prophylactic agent to the subject with or before each dose of the successive doses of the DR IL-18 composition.
5. The method of any one of aspects 2-4, wherein the dose of the CRS prophylactic agent comprises at least one of a nonsteroidal anti-inflammatory drug (NSAID), acetaminophen, histamine H1 antagonist, H2 blocker, or fluids administered to the subject.
6. The method of any one of aspects 2-5, where in the dose of the CRS prophylactic agent comprises an NSAID, optionally wherein the NSAID is an oral NSAID.
7. The method of aspect 6, wherein the dose of the CRS prophylactic agent is administered before the dose, or each dose, of the successive doses of the DR IL-18 composition, optionally at least 1 hour before, optionally 1 to 2 hours before.
8. The method of aspect 6 or 7, wherein the NSAID is further administered for at least 24 hours, optionally at least 48 hours, following the dose, or each dose, of the successive doses of the DR IL-18 composition.
9. The method of any one of aspects 6-8, wherein the NSAID is indomethacin, optionally oral indomethacin.
10. The method of aspect 9, wherein the indomethacin is administered at 50 mg to 100 mg per day, optionally at 75 mg per day.
11. The method of aspect 9 or 10, wherein the indomethacin is administered in 25 mg doses, optionally three times per day.
12. The method of any one of aspects 6-8, wherein the NSAID is ibuprofen, optionally oral ibuprofen.
13. The method of aspect 12, wherein the ibuprofen is administered at 600 mg to 2,400 mg per day, optionally 800 mg to 1,800 mg per day.
14. The method of aspect 12 or 13, wherein the ibuprofen is administered in 200 mg to 600 mg doses, optionally with 6 to 8 hours between ibuprofen doses.
15. The method of any one of aspects 2-5, where in the dose of the CRS prophylactic agent comprises a histamine H1 antagonist, optionally wherein the histamine H1 antagonist is an intravenous histamine H1 antagonist or an oral histamine H1 antagonist.
16. The method of aspect 15, wherein the histamine H1 antagonist is administered before the dose, or each dose, of the successive doses of the DR IL-18 composition, optionally at least 30 minutes before, optionally 30 to 60 minutes before.
17. The method of aspect 15 or 16, wherein the histamine H1 antagonist is diphenhydramine, optionally intravenous diphenhydramine or oral diphenhydramine.
18. The method of aspect 17, wherein 25 mg to 100 mg of diphenhydramine is administered, optionally wherein 50 mg of diphenhydramine is administered.
19. The method of any one of aspects 2-5, where in the dose of the CRS prophylactic agent comprises acetaminophen, optionally wherein the acetaminophen is oral acetaminophen.
20. The method of aspect 19, wherein the acetaminophen is administered before the dose, or each dose, of the successive doses of the DR IL-18 composition, optionally within 2 hours before or 1 hour before.
21. The method of aspect 20, wherein the acetaminophen is further administered for at least 2 days following the dose, or each dose, of the successive doses of the DR IL-18 composition.
22. The method of aspect 19, wherein the acetaminophen is administered within 24 hours of the dose, or each dose, of the successive doses of the DR IL-18 composition.
23. The method of any one of aspects 19-22, wherein 350 mg to 4,000 mg of acetaminophen is administered per day, optionally wherein 650 mg to 3,900 mg of acetaminophen is administered per day.
24. The method of any one of aspects 19-23, wherein the acetaminophen is administered in one or more 650 mg doses, optionally with 4 to 6 hours between doses.
25. The method of any one of aspects 2-5, wherein the dose of the CRS prophylactic agent comprises a H2 blocker, optionally wherein the H2 blocker is an oral H2 blocker or an intravenous H2 blocker.
26. The method of aspect 25, wherein the H2 blocker is administered only before each successive dose of the DR IL-18 composition, optionally at least 30 minutes before each successive dose of the DR IL-18 composition, optionally 30 to 60 minutes before each successive dose of the DR IL-18 composition.
27. The method of aspect 25 or 26, wherein the H2 blocker is famotidine, optionally oral famotidine or intravenous famotidine.
28. The method of aspect 27, wherein 20 mg to 40 mg of oral famotidine is administered.
29. The method of aspect 27, wherein 20 mg of intravenous famotidine is administered.
30. The method of any one of the preceding aspects, wherein the method comprises administering multiple doses of a CRS prophylactic agent to the subject comprising at least a dose of a NSAID and at least a dose of a histamine H1 antagonist; at least a dose of acetaminophen and at least a dose of a histamine H1 antagonist; at least a dose of a NSAID and at least a dose of acetaminophen; or at least a dose of a NSAID, at least a dose of a histamine H1 antagonist, and at least a dose of acetaminophen, optionally wherein the NSAID is selected from indomethacin and ibuprofen and the histamine H1 antagonist is diphenhydramine.
31. The method of aspect 30, further comprising at least a dose of an H2 blocker, optionally wherein the H2 blocker is famotidine, optionally wherein the famotidine is oral famotidine administered at a 20 mg to 40 mg dose or intravenous famotidine administered at a 20 mg dose.
32. The method of aspect 30 or 31, wherein:
   (i) the NSAID is oral indomethacin administered at 50 mg to 100 mg per day, optionally at 75 mg per day, or oral ibuprofen administered at 600 mg to 2,400 mg per day, optionally 800 mg to 1,800 mg per day;
   (ii) the histamine H1 antagonist is intravenous or oral diphenhydramine administered at a dose of 25 mg to 100 mg, optionally 50 mg;
   (iii) the acetaminophen is oral acetaminophen administered at 350 mg to 4,000 mg per day, optionally 650 mg to 3,900 mg per day; or
   (iv) any combination thereof.
33. The method of any one of the preceding aspects, wherein administering the CRS prophylactic agent reduces the display of one or more symptoms of CRS as compared to an equivalent subject having not received the CRS prophylactic agent.
34. The method of any one of the preceding aspects, wherein administering the CRS prophylactic agent reduces the display of one or more symptoms of CRS as compared to a subject receiving an equivalent treatment comprising administration of about 30 μg/kg or more of the DR IL-18 composition without having received the CRS prophylactic agent.
35. The method of any one of the preceding aspects, wherein the subject does not experience grade 4 or higher CRS, optionally wherein the subject does not experience grade 3 or higher CRS.
36. The method of any one of the preceding aspects, wherein each dose of the successive doses of the anti-PD-1 antibody composition is administered to the subject once every three weeks (Q3W).
37. The method of any one of the preceding aspects, wherein each dose of the successive doses of the anti-PD-1 antibody composition is administered to the subject about every 21 days.
38. The method of any one of the preceding aspects, wherein each dose of the successive doses of the anti-PD-1 antibody composition comprises a fixed dose of 200 mg pembrolizumab.
39. The method of any one of the preceding aspects, wherein each dose of the successive doses of the anti-PD-1 antibody composition comprises about 2 mg pembrolizumab per kg body weight of the subject.
40. The method of any one of the preceding aspects, wherein each dose of the successive doses of the anti-PD-1 antibody composition is administered to the subject Q3W as a fixed dose of 200 mg pembrolizumab.
41. The method of any one of the preceding aspects, wherein each dose of the successive doses of the anti-PD-1 antibody composition is administered to the subject about every 21 days as a fixed dose of 200 mg pembrolizumab.
42. The method of any one of the preceding aspects, wherein each dose of the successive doses of the anti-PD-1 antibody composition is administered to the subject Q3W as a dose of about 2 mg pembrolizumab per kg body weight of the subject.
43. The method of any one of the preceding aspects, wherein each dose of the successive doses of the anti-PD-1 antibody composition is administered to the subject about every 21 days as a dose of about 2 mg pembrolizumab per kg body weight of the subject.
44. The method of any one of the preceding aspects, wherein each dose of the successive doses of the DR IL-18 composition is administered to the subject weekly.
45. The method of any one of the preceding aspects, wherein each dose of the successive doses of the DR IL-18 composition is administered to the subject about every 7 days.
46. The method of any one of the preceding aspects, wherein a next dose of the DR IL-18 composition is administered to the subject at least 6 days after a previous dose of the DR IL-18 composition.
47. The method of aspect 46, wherein the next dose of the DR IL-18 composition is administered to the subject at most 9 days after the previous dose of the DR IL-18 composition.
48. The method of any one of the preceding aspects, wherein the polypeptide is a modified IL-18 polypeptide comprising the amino acid sequence set forth in SEQ ID NO:41.
49. The method of any one of the preceding aspects, wherein the polypeptide is a modified IL-18 polypeptide consisting of the amino acid sequence set forth in SEQ ID NO:41.
50. The method of any one of the preceding aspects, wherein the polypeptide is at a concentration of about 30 mg/mL.
51. The method of any one of the preceding aspects, wherein a dose of the DR IL-18 composition comprises at least about 15 μg of the polypeptide per kg body weight of the subject.
52. The method of any one of the preceding aspects, wherein a dose of the DR IL-18 composition comprises at least about 20 μg of the polypeptide per kg body weight of the subject.
53. The method of any one of the preceding aspects, wherein a dose of the DR IL-18 composition comprises at least about 30 μg of the polypeptide per kg body weight of the subject.
54. The method of any one of the preceding aspects, wherein a dose of the DR IL-18 composition comprises at least about 90 μg of the polypeptide per kg body weight of the subject.
55. The method of any one of the preceding aspects, wherein a dose of the DR IL-18 composition comprises at least about 180 μg of the polypeptide per kg body weight of the subject.
56. The method of any one of the preceding aspects, wherein a dose of the DR IL-18 composition comprises at least about 360 μg of the polypeptide per kg body weight of the subject.
57. The method of any one of the preceding aspects, wherein a dose of the DR IL-18 composition comprises at least about 600 μg of the polypeptide per kg body weight of the subject.
58. The method of any one of the preceding aspects, wherein a dose of the DR IL-18 composition comprises at least about 900 μg of the polypeptide per kg body weight of the subject.
59. The method of any one of the preceding aspects, wherein a dose of the DR IL-18 composition comprises at least about 1200 μg of the polypeptide per kg body weight of the subject.
60. The method of any one of aspects 51-53, wherein the dose of the DR IL-18 composition is at least one of an initial dose, a first dose, or a lowest dose administered to the subject.
61. The method of any one of the preceding aspects, wherein each dose of the DR IL-18 composition comprises about 15 μg to about 1200 μg of the polypeptide per kg body weight of the subject.
62. The method of any one of the preceding aspects, wherein a dose of the DR IL-18 composition comprises about 15 μg to about 30 μg, about 30 μg to about 90 μg, about 90 μg to about 180 μg, about 180 μg to about 360 μg, about 360 μg to about 600 μg, about 600 μg to about 900 μg, or about 900 μg to about 1200 μg of the polypeptide per kg body weight of the subject.
63. The method of any one of the preceding aspects, wherein a dose of the DR IL-18 composition comprises about 15 μg, about 30 μg, about 90 μg, about 180 μg, about 360 μg, about 600 μg, about 900 μg, or about 1200 μg of the polypeptide per kg body weight of the subject.
64. The method of any one of the preceding aspects, wherein a subsequent dose of the DR IL-18 composition comprises a greater amount of the polypeptide per body weight of the subject than an amount of the polypeptide administered previously to the subject.
65. The method of aspect 64, wherein the amount of the polypeptide administered previously to the subject was tolerated by the subject.
66. The method of any one of aspects 1-63, wherein a subsequent dose of the DR IL-18 composition comprises a greater amount of the polypeptide per body weight of the subject than an amount of the polypeptide in a previous dose of the DR IL-18 composition.
67. The method of aspect 66, wherein the previous dose was tolerated by the subject.
68. The method of any one of aspects 1-63, wherein a subsequent dose of the DR IL-18 composition comprises a same or similar amount of the polypeptide per body weight of the subject to an amount of the polypeptide administered previously to the subject.
69. The method of aspect 68, wherein the amount of the polypeptide is associated with a recorded Treatment-Emergent Adverse Event in the subject.
70. The method of aspect 69, wherein the recorded Treatment-Emergent Adverse Event is a Grade 1 or Grade 2 Treatment-Emergent Adverse Event according to NCI CTCAE Version 5.0.
71. The method of aspect 69 or 70, wherein the recorded Treatment-Emergent Adverse Event is associated with no limitation to mild limitation in activity of the subject.
72. The method of any one of aspects 69-71, wherein the recorded Treatment-Emergent Adverse Event is associated with providing no or minimal medical intervention, assistance, or therapy to the subject.
73. The method of aspect 68, wherein the amount is a highest tolerable amount.
74. The method of any one of aspects 1-63, wherein a subsequent dose of the DR IL-18 composition comprises a same or similar amount of the polypeptide per body weight of the subject to an amount of the polypeptide in a previous dose of the DR IL-18 composition.
75. The method of aspect 74, wherein the previous dose is associated with causing a recorded Treatment-Emergent Adverse Event in the subject.
76. The method of aspect 75, wherein the recorded Treatment-Emergent Adverse Event is a Grade 1 or Grade 2 Treatment-Emergent Adverse Event according to NCI CTCAE Version 5.0.
77. The method of aspect 75 or 76, wherein the recorded Treatment-Emergent Adverse Event is associated with no limitation to mild limitation in activity of the subject.
78. The method of any one of aspects 75-77, wherein the recorded Treatment-Emergent Adverse Event is associated with providing no or minimal medical intervention, assistance, or therapy to the subject.
79. The method of aspect 74, wherein the previous dose is a highest tolerable dose.
80. The method of any one of aspects 1-63, wherein a subsequent dose of the DR IL-18 composition comprises a lesser amount of the polypeptide per body weight of the subject than an amount of the polypeptide administered previously to the subject.
81. The method of aspect 80, wherein the subject did not tolerate the amount of the polypeptide administered previously.
82. The method of aspect 80 or 81, wherein the amount of the polypeptide administered previously is associated with dose limiting toxicity (DLT).
83. The method of aspect 80, wherein the subsequent dose is a previously tolerated dose.
84. The method of any one of aspects 1-63, wherein a subsequent dose of the DR IL-18 composition comprises a lesser amount of the polypeptide per body weight of the subject than an amount of the polypeptide in a previous dose of the DR IL-18 composition.
85. The method of aspect 84, wherein the previous dose was not tolerated by the subject.
86. The method of aspect 84, wherein the previous dose is associated with dose limiting toxicity (DLT).
87. The method of aspect 84, wherein the subsequent dose is a previously tolerated dose.
88. The method of any one of the preceding aspects, wherein the anti-PD-1 and DR IL-18 compositions are administered to the subject is a series of cycles, wherein each cycle comprises about 21 days.
89. The method of aspect 88, wherein the anti-PD-1 antibody composition is administered once per cycle.
90. The method of aspects 88 or 89, wherein the DR IL-18 composition is administered one, two, or three times during each cycle.
91. The method of any one of aspects 88-90, wherein the series of cycles comprises a current cycle and a next cycle, wherein the next cycle runs consecutively to the current cycle.
92. The method of any one of aspects 88-91, wherein the series of cycles comprises a first cycle, and wherein the first cycle comprises a first day.
93. The method of aspect 92, wherein a dose of the anti-PD-1 antibody composition comprising 200 mg of pembrolizumab is administered to the subject on the first day of the first cycle.
94. The method of aspect 92 or 93, wherein a dose of the DR IL-18 composition comprising between about 15 μg to about 30 μg of the polypeptide per kg of body weight of the subject is administered to the subject on the first day of the first cycle.
95. The method of any one of aspects 88-94, wherein the series of cycles comprises at least 8 cycles.
96. The method of any one of aspects 88-95, wherein the series of cycles comprises no more than 35 cycles.
97. The method of any one of the preceding aspects, wherein the anti-PD-1 and DR IL-18 compositions are administered for at least 24 weeks.
98. The method of any one of the preceding aspects, wherein the anti-PD-1 and DR IL-18 compositions are administered until the subject has been administered at least 8 doses of the anti-PD-1 antibody composition.
99. The method of any one of the preceding aspects, wherein the anti-PD-1 and DR IL-18 compositions are administered until the subject exhibits a complete response (CR) and has been treated for at least 24 weeks or has been administered at least 8 doses of the anti-PD-1 antibody composition.
100. The method of any one of aspects 88-96, wherein the anti-PD-1 and DR IL-18 compositions are administered until the subject exhibits a complete response (CR) and has been treated for at least 8 cycles.
101. The method of any one of the preceding aspects, wherein the anti-PD-1 and DR IL-18 compositions are administered until the subject exhibits a complete response (CR) and has been administered at least 8 doses of the anti-PD-1 antibody composition.
102. The method of any one of the preceding aspects, wherein the anti-PD-1 and DR IL-18 compositions are administered until the subject has been administered at least about 24 doses of the DR IL-18 composition.
103. The method of any one of the preceding aspects, wherein the anti-PD-1 and DR IL-18 compositions are administered until the subject exhibits an intervention-related toxicity specified as a reason for permanent discontinuation.
104. The method of aspect 103, wherein the intervention-related toxicity is at least one of a Grade 4, Grade 3, or recurrent Grade 2 Treatment-Emergent Adverse Event according to NCI CTCAE Version 5.0.
105. The method of aspect 104, wherein the Treatment-Emergent Adverse Event is associated with marked or severe limitation to extreme limitation in activity of the subject.
106. The method of aspect 104 or 105, wherein the Treatment-Emergent Adverse Event is life threatening or requires significant medical intervention or hospitalization.
107. The method of any one of the preceding aspects, wherein the anti-PD-1 and DR IL-18 compositions are administered for at most about 2 years.
108. The method of any one of the preceding aspects, wherein at most 35 doses of the anti-PD-1 antibody composition are administered to the subject.
109. The method of any one of the preceding aspects, wherein a dose of the anti-PD-1 antibody composition is administered to the subject at least about 60 minutes before a dose of the DR IL-18 composition is administered to the subject.
110. The method of any one of the preceding aspects, wherein a dose of the DR IL-18 composition is administered to the subject at least 60 minutes after a dose of the anti-PD-1 antibody composition is administered to the subject.
111. The method of any one of the preceding aspects, wherein (a) and (b) are performed sequentially.
112. The method of any one of the preceding aspects, wherein (a) is performed before (b).
113. The method of any one of aspects 1-108, wherein (b) is performed before (a).
114. The method of any one of aspects 1-108, wherein (a) and (b) are performed simultaneously.
115. The method of any one of the preceding aspects, wherein a dose of the anti-PD-1 antibody composition is administered to the subject by intravenous infusion.
116. The method of any one of the preceding aspects, wherein each dose of the anti-PD-1 antibody composition is administered to the subject by intravenous infusion.
117. The method of any one of the preceding aspects, wherein a dose of the DR IL-18 composition is administered to the subject by subcutaneous injection.
118. The method of any one of the preceding aspects, wherein each dose of the DR IL-18 composition is administered to the subject by subcutaneous injection.
119. The method of any one of the preceding aspects, wherein the disease comprises cancer or a solid tumor.
120. The method of aspect 119, wherein the cancer or solid tumor is associated with melanoma, non-small cell lung cancer (NSCLC), small cell lung cancer, head and neck squamous cell cancer (HNSCC), classical Hodgkin Lymphoma (cHL), primary mediastinal large B-cell lymphoma (PMBCL), urothelial carcinoma, microsatellite instability-high (MSI-H) or mismatch repair deficient cancer (dMMR) cancer, microsatellite instability-high or mismatch repair deficient colorectal cancer (CRC), colorectal cancer, gastric cancer, esophageal or gastroesophageal junction cancer, esophageal or gastroesophageal junction adenocarcinoma, locally advanced or metastatic esophageal or gastroesophageal junction (GEJ) carcinoma, malignant pleural mesothelioma, cervical cancer, ovarian cancer, hepatocellular carcinoma (HCC), Merkel cell carcinoma (MCC), kidney cancer, renal cell carcinoma (RCC), bladder cancer, endometrial carcinoma, liver cancer, tumor mutational burden-high (TMB-H) cancer, cutaneous squamous cell carcinoma (cSCC), triple-negative breast cancer (TNBC), or any combination thereof.
121. The method of aspect 120, wherein the cancer or solid tumor is resistant to PD-1 checkpoint inhibitors.
122. The method of aspect 119, wherein the cancer or solid tumor is associated with Melanoma, Merkel cell, RCC, urothelial, NSCLC (with no epidermal growth factor receptor, TRK receptor, or anaplastic lymphoma kinase positive mutations/fusions), TNBC, SCCHN, MSI-H, TMB-H or mismatch repair deficient, gastric, cervical, endometrial, cutaneous squamous, small cell lung, esophageal, HCC, or any combination thereof, and wherein the cancer or solid tumor is resistant to PD-1 checkpoint inhibitors.
123. The method of aspect 119, wherein the cancer or solid tumor is associated with platinum-resistant ovarian cancer or microsatellite stable colorectal cancer.
124. The method of aspect 119, wherein the cancer is a hematological cancer.
125. The method of aspect 124, wherein the hematological cancer is selected from the group consisting of: a leukemia, a lymphoma, a myelodysplastic syndrome, a myeloproliferative disorder, and myeloma.
126. The method of aspect 125, wherein the hematological cancer is a myeloma.
127. The method of aspect 125, wherein the hematological cancer is a lymphoma, optionally B cell lymphoma.
128. The method of aspect 125, wherein the hematological cancer is a leukemia, optionally acute myeloid leukemia.
129. The method of any one of the preceding aspects, comprising administering one or more doses of a CRS prophylactic agent to the subject with or before each dose of the successive doses of the DR IL-18 composition, wherein the CRS prophylactic agent comprises at least one of a NSAID, a histamine H1 antagonist, acetaminophen, or a H2 blocker, and wherein:
   (i) the polypeptide is a modified IL-18 polypeptide comprising or consisting of the amino acid sequence set forth in SEQ ID NO:41;
   (ii) the dose of the DR IL-18 composition comprises at least about 30 μg of the polypeptide per kg body weight of the subject;
   (iii) the dose of the anti-PD-1 antibody composition comprises at least about 200 mg of pembrolizumab; and
   (iv) the disease comprises a PD-1 checkpoint inhibitor resistant solid tumor selected from the group consisting of: melanoma, Merkel cell, RCC, urothelial, NSCLC, TNBC, SCCHN, MSI-H tumor, TMB-H or mismatch repair deficient tumor, gastric, cervical, endometrial, cutaneous squamous, small cell lung, esophageal, HCC, or any combination thereof; or platinum-resistant ovarian cancer or microsatellite stable colorectal cancer.
130. A method of treating a disease in a human patient comprising administering to the patient an anti-PD-1 antibody, or antigen binding fragment thereof, in combination with a decoy-resistant (DR) IL-18 composition comprising a polypeptide to the subject, wherein the polypeptide is a modified IL-18 polypeptide comprising an amino acid sequence set forth in any one of SEQ ID NOs: 36-43, wherein the anti-PD-1 antibody, or antigen binding fragment thereof, comprises a light chain complementarity determining regions (CDRs) comprising a sequence of amino acids as set forth in SEQ ID NOs: 1, 2 and 3 and heavy chain CDRs comprising a sequence of amino acids as set forth in SEQ ID NOs: 6, 7 and 8.
131. The method of aspect 130, wherein the anti-PD-1 antibody, or antigen binding fragment thereof, is an anti-PD-1 monoclonal antibody.
132. The method of aspect 130 or 131, wherein the anti-PD-1 antibody, or antigen binding fragment thereof, is administered to the patient at a dose of about 200 mg once every three weeks.
133. The method of any one of aspects 130-132, wherein the anti-PD-1 antibody, or antigen binding fragment thereof, is administered to the patient at a dose of about 400 mg once every six weeks.
134. The method of any one of aspects 130-133, wherein the anti-PD-1 antibody, or antigen binding fragment thereof, is administered to the patient by an IV infusion.
135. The method of aspect 134, wherein the anti-PD-1 antibody, or antigen binding fragment thereof, is administered to the patient by an IV infusion over about 30 minutes on day 1 of each treatment cycle.
136. The method of any one of aspects 130-135, wherein the anti-PD-1 antibody, or antigen binding fragment thereof, is pembrolizumab.
137. The method of any one of aspects 130-135, wherein the anti-PD-1 monoclonal antibody is a pembrolizumab variant.
138. The method of any one of aspects 130-137, wherein the anti-PD-1 antibody, or antigen-binding fragment thereof, is administered as part of a composition, wherein the composition comprises 130 mg/mL of the anti-PD-1 antibody, or antigen-binding fragment thereof.
139. The method of any one of aspects 130-137, wherein the anti-PD-1 antibody, or antigen-binding fragment thereof, is administered as part of a composition, wherein the composition comprises 165 mg/mL of the anti-PD-1 antibody, or antigen-binding fragment thereof.
140. The method of any one of aspects 130-139, the DR IL-18 composition is administered to the patient weekly.
141. The method of any one of aspects 130-140, wherein the DR IL-18 composition is administered to the patient about every 7 days.
142. The method of any one of aspects 130-141, wherein doses of the DR IL-18 composition are administered to the patient at least 6 days apart.
143. The method of aspect 142, wherein doses of the DR IL-18 composition are administered to the patient at most 9 days apart.
144. The method of any one of aspects 130-143, wherein the polypeptide is a modified IL-18 polypeptide comprising the amino acid sequence set forth in SEQ ID NO:41.
145. The method of any one of aspects 130-144, wherein the polypeptide is a modified IL-18 polypeptide consisting of the amino acid sequence set forth in SEQ ID NO:41.
146. The method of any one of aspects 130-145, wherein the polypeptide is at a concentration of about 30 mg/mL.
147. The method of any one of aspects 130-146, wherein the DR IL-18 composition comprises about 15 μg to about 1200 μg of the polypeptide per kg body weight of the patient.
148. The method of any one of aspects 130-147, wherein the DR IL-18 composition is administered to the patient at a dose of about 15 μg to about 30 μg, about 30 μg to about 90 μg, about 90 μg to about 180 μg, about 180 μg to about 360 μg, about 360 μg to about 600 μg, about 600 μg to about 900 μg, or about 900 μg to about 1200 μg of the polypeptide per kg body weight of the patient.
149. The method of any one of aspects 130-148, wherein the DR IL-18 composition is administered to the patient at a dose of at least about 30 μg of the polypeptide per kg body weight of the patient.
150. The method of any one of aspects 130-149, wherein the DR IL-18 composition is administered to the patient by subcutaneous injection.
151. The method of any one of aspects 130-150, wherein the disease comprises cancer or a solid tumor.
152. The method of aspect 151, wherein the cancer or solid tumor is resistant to PD-1 checkpoint inhibitors.
153. The method of aspect 151 or 152, wherein the cancer or solid tumor is, or is associated with, melanoma, non-small cell lung cancer (NSCLC), small cell lung cancer, head and neck squamous cell cancer (HNSCC), classical Hodgkin Lymphoma (cHL), primary mediastinal large B-cell lymphoma (PMBCL), urothelial carcinoma, microsatellite instability-high (MSI-H) or mismatch repair deficient cancer (dMMR) cancer, microsatellite instability-high or mismatch repair deficient colorectal cancer (CRC), colorectal cancer, gastric cancer, esophageal or gastroesophageal junction cancer, esophageal or gastroesophageal junction adenocarcinoma, locally advanced or metastatic esophageal or gastroesophageal junction (GEJ) carcinoma, malignant pleural mesothelioma, cervical cancer, ovarian cancer, hepatocellular carcinoma (HCC), Merkel cell carcinoma (MCC), kidney cancer, renal cell carcinoma (RCC), bladder cancer, endometrial carcinoma, liver cancer, tumor mutational burden-high (TMB-H) cancer, cutaneous squamous cell carcinoma (cSCC), triple-negative breast cancer (TNBC), or any combination thereof.
154. The method of aspect 153, wherein the cancer or solid tumor is, or is associated with, melanoma, Merkel cell, RCC, urothelial, NSCLC (with no epidermal growth factor receptor, TRK receptor, or anaplastic lymphoma kinase positive mutations/fusions), TNBC, SCCHN, MSI-H, TMB-H or mismatch repair deficient, gastric, cervical, endometrial, cutaneous squamous, small cell lung, esophageal, HCC, or any combination thereof, and wherein the cancer or solid tumor is resistant to PD-1 checkpoint inhibitors.
155. The method of aspect 151, wherein the cancer or solid tumor is, or is associated with, platinum-resistant ovarian cancer or microsatellite stable colorectal cancer.
156. The method of aspect 151, wherein the cancer is a hematological cancer.
157. The method of aspect 156, wherein the hematological cancer is selected from the group consisting of: a leukemia, a lymphoma, a myelodysplastic syndrome, a myeloproliferative disorder, and myeloma.
158. The method of aspect 157, wherein the hematological cancer is a myeloma.
159. The method of aspect 157, wherein the hematological cancer is a lymphoma, optionally B cell lymphoma.
160. The method of aspect 157, wherein the hematological cancer is a leukemia, optionally acute myeloid leukemia.
161. The method of any one of aspects 130-160, further comprising administering a cytokine release syndrome (CRS) prophylactic agent to the patient.
162. The method of aspect 161, wherein the CRS prophylactic agent is administered with or before the DR IL-18 composition.
163. The method of aspect 161 or 162, wherein the CRS prophylactic agent comprises at least one of a nonsteroidal anti-inflammatory drug (NSAID), acetaminophen, histamine H1 antagonist, or H2 blocker.
164. The method of any one of aspects 161-163, where in the CRS prophylactic agent comprises an NSAID, optionally wherein the NSAID is an oral NSAID.
165. The method of aspect 164, wherein the CRS prophylactic agent is administered before the DR IL-18 composition, optionally at least 1 hour before, optionally 1 to 2 hours before.
166. The method of aspect 164 or 165, wherein the NSAID is further administered for at least 24 hours, optionally at least 48 hours, following the DR IL-18 composition.
167. The method of any one of aspects 164-166, wherein the NSAID is indomethacin, optionally oral indomethacin.
168. The method of aspect 167, wherein the indomethacin is administered at 50 mg to 100 mg per day, optionally at 75 mg per day.
169. The method of aspect 167 or 168, wherein the indomethacin is administered in 25 mg doses, optionally three times per day.
170. The method of any one of aspects 164-166, wherein the NSAID is ibuprofen, optionally oral ibuprofen.
171. The method of aspect 170, wherein the ibuprofen is administered at 600 mg to 2,400 mg per day, optionally 800 mg to 1,800 mg per day.
172. The method of aspect 170 or 171, wherein the ibuprofen is administered in 200 mg to 600 mg doses, optionally with 6 to 8 hours between ibuprofen doses.
173. The method of any one of aspects 161-163, where in the CRS prophylactic agent comprises a histamine H1 antagonist, optionally wherein the histamine H1 antagonist is an intravenous histamine H1 antagonist or an oral histamine H1 antagonist.
174. The method of aspect 173, wherein the histamine H1 antagonist is administered before the DR IL-18 composition, optionally at least 30 minutes before, optionally 30 to 60 minutes before.
175. The method of aspect 173 or 174, wherein the histamine H1 antagonist is diphenhydramine, optionally intravenous diphenhydramine or oral diphenhydramine.
176. The method of aspect 175, wherein 25 mg to 100 mg of diphenhydramine is administered, optionally wherein 50 mg of diphenhydramine is administered.
177. The method of any one of aspects 161-163, where in the CRS prophylactic agent comprises acetaminophen, optionally wherein the acetaminophen is oral acetaminophen.
178. The method of aspect 177, wherein the acetaminophen is administered before the DR IL-18 composition, optionally within 2 hours before or 1 hour before.
179. The method of aspect 178, wherein the acetaminophen is further administered for at least 2 days following the DR IL-18 composition.
180. The method of aspect 179, wherein the acetaminophen is administered within 24 hours of the DR IL-18 composition.
181. The method of any one of aspects 177-180, wherein 350 mg to 4,000 mg of acetaminophen is administered per day, optionally wherein 650 mg to 3,900 mg of acetaminophen is administered per day.
182. The method of any one of aspects 177-181, wherein the acetaminophen is administered in one or more 650 mg doses, optionally with 4 to 6 hours between doses.
183. The method of any one of aspects 161-163, wherein the CRS prophylactic agent comprises a H2 blocker, optionally wherein the H2 blocker is an oral H2 blocker or an intravenous H2 blocker.
184. The method of aspect 183, wherein the H2 blocker is administered only before the DR IL-18 composition, optionally at least 30 minutes before the DR IL-18 composition, optionally 30 to 60 minutes before the DR IL-18 composition.
185. The method of aspect 183 or 184, wherein the H2 blocker is famotidine, optionally oral famotidine or intravenous famotidine.
186. The method of aspect 185, wherein 20 mg to 40 mg of oral famotidine is administered.
187. The method of aspect 185, wherein 20 mg of intravenous famotidine is administered.
188. The method of any one of aspects 161-187, wherein the method comprises administering multiple CRS prophylactic agents to the patient comprising at least a NSAID and at least a histamine H1 antagonist; at least acetaminophen and at least a histamine H1 antagonist; at least a NSAID and at least acetaminophen; or at least a NSAID, at least a histamine H1 antagonist, and at least acetaminophen, optionally wherein the NSAID is selected from indomethacin and ibuprofen and the histamine H1 antagonist is diphenhydramine.
189. The method of aspect 188, further comprising at least an H2 blocker, optionally wherein the H2 blocker is famotidine, optionally wherein the famotidine is oral famotidine administered at a 20 mg to 40 mg dose or intravenous famotidine administered at a 20 mg dose.
190. The method of aspect 188 or 189, wherein:
   (i) the NSAID is oral indomethacin administered at 50 mg to 100 mg per day, optionally at 75 mg per day, or oral ibuprofen administered at 600 mg to 2,400 mg per day, optionally 800 mg to 1,800 mg per day;
   (ii) the histamine H1 antagonist is intravenous or oral diphenhydramine administered in a dose of 25 mg to 100 mg, optionally 50 mg;
   (iii) the acetaminophen is oral acetaminophen administered at 350 mg to 4,000 mg per day, optionally 650 mg to 3,900 mg per day; or
   (iv) any combination thereof.
191. The method of any one of aspects 161-190, wherein administering the CRS prophylactic agent reduces the display of one or more symptoms of CRS as compared to an equivalent patient without receiving the CRS prophylactic agent.
192. The method of any one of aspects 161-191, wherein administering the CRS prophylactic agent reduces the display of one or more symptoms of CRS as compared to a patient receiving an equivalent treatment comprising administration of about 30 μg/kg or more of the DR IL-18 composition without having received the CRS prophylactic agent.
193. The method of any one of aspects 161-192, wherein the patient does not experience grade 4 or higher CRS, optionally wherein the subject does not experience grade 3 or higher CRS.
194. The method of any one of aspects 130-193, comprising administering one or more CRS prophylactic agents to the patient with or before the DR IL-18 composition, wherein the one or more CRS prophylactic agents comprise a NSAID, a histamine H1 antagonist, acetaminophen, a H2 blocker, or a combination thereof, and wherein:
   (i) the polypeptide is a modified IL-18 polypeptide comprising or consisting of the amino acid sequence set forth in SEQ ID NO:41;
   (ii) the dose of the DR IL-18 composition comprises at least about 30 μg of the polypeptide per kg body weight of the patient;
   (iii) the dose of the anti-PD-1 antibody composition comprises at least about 200 mg of pembrolizumab or a pembrolizumab variant; and
   (iv) the disease comprises a PD-1 checkpoint inhibitor resistant solid tumor selected from the group consisting of: melanoma, Merkel cell, RCC, urothelial, NSCLC, TNBC, SCCHN, MSI-H tumor, TMB-H or mismatch repair deficient tumor, gastric, cervical, endometrial, cutaneous squamous, small cell lung, esophageal, HCC, or any combination thereof; or platinum-resistant ovarian cancer or microsatellite stable colorectal cancer.
195. The method of any one of aspects 130-194, wherein the disease is a cancer or a tumor and the method thereby causes one or more improvements in the patient's cancer or tumor.
196. The method of aspect 195, wherein the one or more improvements comprise stable disease for at least 12 weeks.
197. The method of aspect 195 or 196, wherein the one or more improvements comprise an immunotherapy-induced regression of the cancer or tumor in the patient.
198. The method of any one of aspects 195-197, wherein the one or more improvements comprise a partial response as assessed by RECIST Version 1.1.
199. The method of any one of aspects 195-198, wherein the one or more improvements comprise a complete response as assessed by RECIST Version 1.1.
200. Use of an anti-PD-1 antibody, or antigen binding fragment thereof, in a method of treating cancer, according to any one of aspects 130-199.
201. A method of treating cancer, comprising administering to a human patient in need thereof pembrolizumab and a decoy-resistant IL-18 polypeptide.
202. The method of aspect 201, wherein the patient is administered about 200 mg of pembrolizumab once every three weeks.
203. The method of aspect 201, wherein the patient is administered about 400 mg of pembrolizumab once every six weeks.

### EXAMPLES

### Example #1: Pembrolizumab Combination Therapy with DR IL-18 Polypeptide

A phase Ia and 2 study for safety, preliminary efficacy, PK and PD of DR IL-18 polypeptide and pembrolizumab is conducted in various condition/diseases including cancer, solid tumor, melanoma, renal cell carcinoma, triple negative breast cancer, non-small cell lung cancer, squamous cell carcinoma of the head and neck, carcinoma, and MSI-H. An arm present in the Phase I clinical trial is for combination therapy of pembrolizumab and DR IL-18 polypeptide. Phase I dose escalation in combination with pembrolizumab will start at a dose of 30 μg/kg of DR IL-18 polypeptide and 200 mg every 3 weeks of pembrolizumab. Patients will be treated every week with DR IL-18 polypeptide and every three weeks with pembrolizumab. The maximum tolerated dose (MTD) will be determined based on the modified toxicity probability interval (mTIP) design.

In an exemplary embodiment, a subject is administered pembrolizumab by intravenous infusion (200 mg Q3W) and decoy-resistant IL-18 polypeptide by subcutaneous injection (30 μg/kg subject's body weight QW) for at most about 2 years to treat tumors associated with non-small cell lung cancer. The tumors would shrink throughout the course of the treatment, resulting in significant reductions in tumor size and tumor number until the patient exhibits signs of complete disease remission after being administered the combination treatment for 2 years.

### Example #2: CRS Prophylaxis in Patients Treated with DR IL-18 Polypeptide Plus Pembrolizumab Combination Therapy

A risk of CRS when DR IL-18 polypeptide is administered subcutaneously as a monotherapy has been observed. All patients are now given prophylaxis before each injection.

Consensus American Society for Transplantation and Cellular Therapy (ASTCT) grading for CRS is as follows:

| CRS | | | | |
|---|---|---|---|---|
| Parameter | Grade 1 | Grade 2 | Grade 3 | Grade 4 |
| Fever | Temp. ≥38 deg. C. | Temp. ≥38deg. C. | Temp. ≥38 deg. C. | Temp. ≥38 deg. C. |
| | | | With | |
| Hypotension | None | Not | Requiring a | Requiring multiple |
| | | requiring | vasopressor with or | vasopressors (excluding |
| | | vasopressors | without vasopressin | vasopressin) |
| | | | And/or | |
| Hypoxia | None | Requiring | Requiring high-flow | Requiring positive |
| | | low-flow | nasal-canula, | pressure (e.g., CPAP, |
| | | nasal canula | facemask, | BiPAP, intubation and |
| | | or blow-by | nonrebreather mask, | mechanical ventilation) |
| | | | or Venturi mask | |

Abbreviations: BiPAP = bilevel positive airway pressure; CPAP = continuous positive airway pressure.
See also e.g., Lee et al. Biology of Blood and Marrow Transplantation. 25 (2019) 625-639

A three-patient cohort, that previously experienced an insufficient response to PD-1 checkpoint inhibitor therapy, received combination therapy of weekly subcutaneous DR IL-18 polypeptide at 30 μg/kg plus 200 mg pembrolizumab intravenously every three weeks. DR IL-18 polypeptide was administered with CRS prophylaxis, given with each dose, that included administration of (a) an oral nonsteroidal anti-inflammatory drug (NSAID) 1-2 hours prior to DR IL-18 polypeptide dosing and continuing for at least 48 hours post-dose (using indomethacin at 25 mg 3 times daily or oral ibuprofen at 200 mg to 600 mg every 6 to 8 hours); (b) intravenous or oral diphenhydramine at 50 mg (or comparable dose of next-generation histamine H1 antagonist) 30-60 minutes prior to dosing with DR IL-18 polypeptide; (c) 650 mg oral acetaminophen every 4 to 6 hours as needed for the first 24 hours post-dose; and (d) if indicated after initial DR IL-18 polypeptide dosing, famotidine (or other H2 blocker) at 20 mg intravenously or 20-40 mg orally 30-60 minutes prior to subsequent DR IL-18 polypeptide dosing. If not contraindicated by vital signs post-dose, any blood pressure medications were withheld for 24 hours before and at least 48 hours after DR IL-18 polypeptide dosing. For subjects with contraindications to NSAIDs, 650 mg oral acetaminophen every 4-6 hours beginning 1 hour prior to DR IL-18 polypeptide dosing was used and continued for at least 48 hours post-dose. For subjects with or at risk for hypotension, up to 3 liters of oral and/or intravenous fluids were administered within 24 hours post-dose.

All patients in the cohort remained on study through the dose limiting toxicity (DLT) period (i.e., through 21 days with 3 doses of DR IL-18 polypeptide). No patient in the cohort experienced grade 3 or higher CRS and no patients in the cohort required steroid administration for CRS.

### Example #3: Efficacy in Patients Treated with DR IL-18 Polypeptide Plus Pembrolizumab Combination Therapy

Cohorts of patients, as described below, are administered (with CRS prophylaxis given with each dose as described in Example #3) weekly subcutaneous DR IL-18 polypeptide at 30 μg/kg, 60 μg/kg, or other recommended dose (such as the recommended phase 2 dose obtained from DR IL-18 polypeptide dose finding studies) plus 200 mg pembrolizumab intravenously every three weeks. On days that the subject will receive both drugs, pembrolizumab is administered first and DR IL-18 polypeptide is administered, with appropriate CRS prophylaxis, after the pembrolizumab infusion.

Subjects in the cohort will include subjects, with histologically or cytologically confirmed diagnosis of advanced/metastatic melanoma, Merkel cell carcinoma, renal cell carcinoma (RCC), urothelial, non-small cell lung cancer (NSCLC), triple-negative breast cancer (TNBC), squamous cell carcinoma of head and neck (SCCHN), any microsatellite instability high (MSI-H), any high tumor mutation burden (TMB-H) or mismatch repair deficient, gastric, cervical, endometrial, cutaneous squamous, small cell lung, esophageal, and hepatocellular carcinoma cancer (HCC) cancer/tumor(s), which experienced an insufficient response to a checkpoint inhibitor programmed cell death receptor-1 (PD-1) therapy. TNBC is diagnosed in a tumor which does not express estrogen receptor or progesterone receptor, is not human epidermal growth factor receptor 2 (HER2) 3+ on immunohistochemistry (IHC) or is negative by fluorescence in situ hybridization (FISH). MSI high tumors have mutations in 30% or more microsatellites by PCR or are negative for MSH1/2/6 or PMS-2 by IHC. TMB-H high tumor has 10 mutations per megabase (mut/Mb) or greater calculated from whole genome sequencing or whole exome sequencing. The following tumors are allowed in expansion cohorts: platinum resistant ovarian cancer and microsatellite stable colorectal cancer.

Disease assessments will be made using a computed tomography (CT) or magnetic resonance imaging (MRI) scan of the chest, abdomen, and pelvis (and other relevant areas if they include target lesions). Determinations of the magnitude and duration of changes in tumor size will be based on well-established response and progression criteria (Response Evaluation Criteria in Solid Tumors (RECIST) Version 1.1; see Eisenhauer et al. (2009) European Journal of Cancer 45:228-247) as applied to radiographic measurements. Disease assessments will be done every 6 weeks for the first 24 weeks then every 12 weeks. Clinical laboratory, pharmacodynamic, pharmacogenomic, pharmacokinetic, and other assessments will also be performed.

The cohort is expected to display evidence of pharmacodynamic activity as well as substantial evidence of tumor regressions including objective responses (CR and PR) as assessed by RECIST Version 1.1. Stable disease for ≥12 weeks will be considered clinically relevant in this population of patients.

### Example #4: DR IL-18 Polypeptide and Anti-PD-1 ICI Antibody Combination Therapy Reduces Tumor Growth in CT26 Murine Colorectal Tumor Model

A CT26 murine colorectal cancer model was used to evaluate the anti-tumor activity in colorectal cancer of decoy resistant (DR) IL-18 polypeptide, alone and in combination with an anti-PD-1 immune checkpoint inhibitor (ICI). CT26 is an N-nitroso-N-methylurethane-(NNMU) induced undifferentiated colon carcinoma cell line established from BALB/c mice with aggressive colon carcinoma. CT26 is considered a “warm” tumor model, meaning it generally resides between an immunogenic “hot” and a nonimmunogenic “cold” tumor.

To establish the models, CT26 cells were transplanted into host mice as a xenograft and allowed to form tumors. Mice were treated with DR IL-18 polypeptide alone (dosed five times by intraperitoneal injection (IP) at 0.32 milligrams per kilogram (mpk) biweekly (BIW), last dosed on study day 15), anti-PD-1 antibody alone (dosed 10 mpk BIW for 5 doses) or with DR IL-18 polypeptide in combination with anti-PD-1 (dosed the same as each corresponding monotherapy). Untreated “vehicle” controls were dosed with delivery vehicle only (i.e., without DR IL-18 polypeptide or any ICI). As an assay for cancer growth, tumor size was measured (in cubic millimeters (mm3)) in each animal over time during the course of the study.

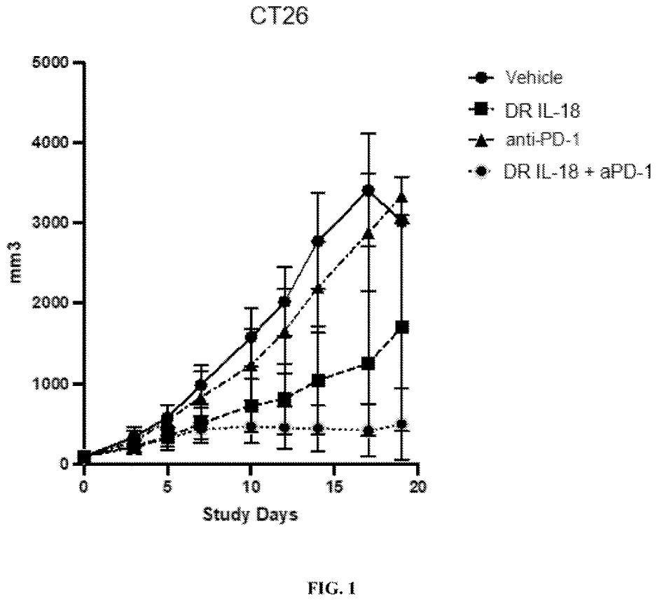

As can be seen in FIG. 1, tumors grew rapidly in untreated vehicle control mice and mice administered anti-PD-1 monotherapy. Tumor growth was substantially reduced in mice treated with DR IL-18 polypeptide monotherapy as compared to vehicle control mice and anti-PD-1 monotherapy treated mice. However, tumor growth in mice treated with DR IL-18 polypeptide and anti-PD-1 combination was substantially reduced, if not halted, as compared to either of the monotherapy treatment conditions. Accordingly, this example demonstrates that combination therapy that includes DR IL-18 polypeptide in combination with an anti-PD-1 antibody ICI has effective anti-tumor activity, greatly reducing, if not halting, colorectal tumor growth. The anti-tumor activity of the combination therapy was substantially greater than either DR IL-18 polypeptide monotherapy or anti-PD-1 ICI monotherapy.

### Example #5: DR IL-18 Polypeptide and Anti-PD-1 ICI Antibody Combination Therapy Reduces Tumor Growth in MC38 Murine Colorectal Tumor Model

A MC38 murine colorectal cancer model was used to evaluate the anti-tumor activity in colorectal cancer of DR IL-18 polypeptide, alone and in combination with an anti-PD-1 ICI. The MC38 tumorigenic epithelial cell line is isolated from mice with colon adenocarcinoma and expresses high levels of human carcinoembryonic antigen (CEA). MC38 contains a high mutational burden and is sensitive to immune checkpoint immunotherapy.

To establish the models, MC38 cells were transplanted into host mice as a xenograft and allowed to form tumors. Mice were treated with DR IL-18 polypeptide alone (dosed five times IP at 0.32 mpk BIW, last dosed on study day 15), anti-PD-1 antibody alone (dosed 10 mpk BIW for 5 doses), or with DR IL-18 polypeptide in combination with anti-PD-1 (dosed the same as each corresponding monotherapy). Untreated “vehicle” controls were dosed with delivery vehicle only (i.e., without DR IL-18 polypeptide or any ICI). As an assay for cancer growth, tumor size was measured (in mm3) in each animal over time during the course of the study.

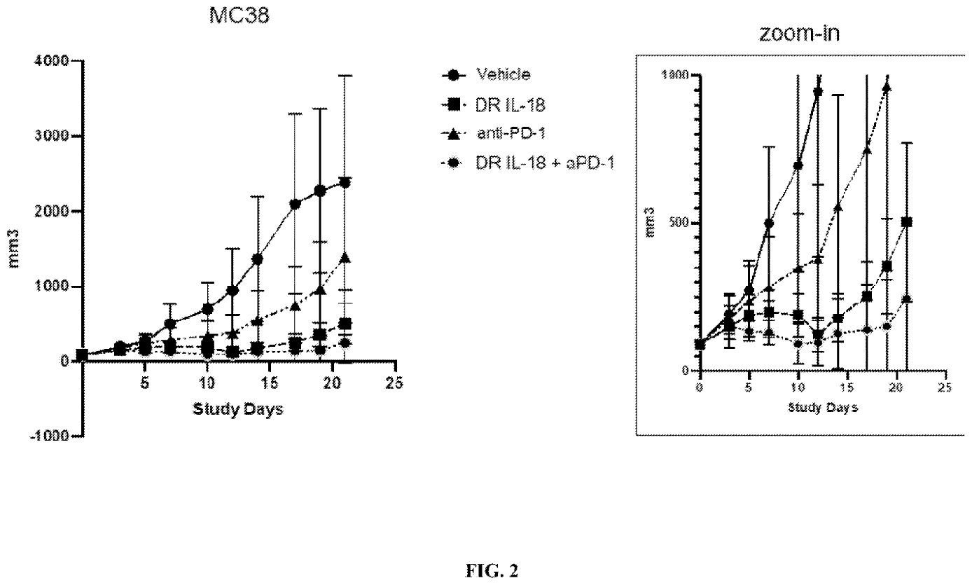

As can be seen in FIG. 2, tumors grew, on average, to over 2000 mm³ in untreated vehicle control mice. Mice administered anti-PD-1 monotherapy showed a reduction in MC38 tumor growth as compared to vehicle control. Tumor growth was reduced in mice treated with DR IL-18 polypeptide monotherapy as compared to anti-PD-1 monotherapy treated mice. However, MC38 tumor growth more substantially reduced, as compared with either monotherapy, in mice treated with DR IL-18 polypeptide and anti-PD-1 combination. Accordingly, this example demonstrates that combination therapy that includes DR IL-18 polypeptide in combination with an anti-PD-1 antibody ICI has effective anti-colorectal tumor activity that is superior to both DR IL-18 polypeptide monotherapy and anti-PD-1 ICI monotherapy.

### Example #6: DR IL-18 Polypeptide and Anti-PD-1 ICI Antibody Combination Therapy Reduces Tumor Growth and Improves Therapeutic Response in Murine Heme Tumor Models

Various heme tumor murine models, including MPC-11 (myeloma), A-20 (B cell lymphoma), and C1498 (acute myeloid leukemia), were used to evaluate the anti-tumor activity in blood cancers of DR IL-18 polypeptide, alone and in combination with an anti-PD-1 ICI. MPC-11 (Merwin Plasma Cell tumor-11) is a mouse plasma cell myeloma with a H-2d haplotype that is commercially available and commonly used for anti-cancer immunotherapeutic efficacy and other studies. A-20 is a cell line derived from a spontaneous mouse reticulum cell sarcoma that is commercially available and commonly used for B cell lymphoma studies including anti-cancer immunotherapeutic efficacy and other studies. C1498 is an aggressive acute myeloid leukemia (AML) cell line which originated spontaneously in a C57BL/6 mouse that is commercially available and commonly used for AML studies including anti-cancer immunotherapeutic efficacy and other studies.

To establish the models, MPC-11, A-20, or C1498 cells were transplanted into host mice as xenografts and allowed to engraft, forming tumors. Mice were treated with DR IL-18 polypeptide alone (dosed five times IP at 0.32 mpk BIW, last dosed on study day 15), anti-PD-1 antibody alone (dosed 10 mpk BIW for 5 doses), or with DR IL-18 polypeptide in combination with anti-PD-1 (dosed the same as each corresponding monotherapy). Untreated “vehicle” controls were dosed with delivery vehicle only (i.e., without DR IL-18 polypeptide or any ICI).

Both DR IL-18 polypeptide monotherapy and DR IL-18 polypeptide plus anti-PD-1 antibody ICI combination therapy demonstrated reductions in tumor growth as compared to vehicle controls and anti-PD-1 antibody ICI monotherapy. In addition to tumor growth inhibition, mice were also evaluated for treatment response using a pseudo-clinical scoring criteria that correlated with scoring used for partial response (PR) and complete response (CR) in human clinical trials. In the A-20 tumor model, improvements in response were observed in mice treated with combination therapy (DR IL-18 polypeptide plus anti-PD-1 antibody ICI) as compared to DR IL-18 polypeptide monotherapy. Treatment with DR IL-18 polypeptide monotherapy resulted in 60% CR and 40% PR, whereas treatment with combination therapy resulted in 87.5% CR and 12.5% PR. Collectively, this example demonstrates that combining DR IL-18 polypeptide with anti-PD-1 ICI therapy has effective anti-heme (e.g., anti-myeloma, anti-B cell lymphoma, and anti-AML) tumor activity that is superior to monotherapy with anti-PD-1 ICI and results in treatment responses that are improved as compared to DR IL-18 polypeptide monotherapy.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Amino Acid Sequences

SEQ ID NO: 35
wildtype IL-18
YFGKLESKLSVIRNLNDQVLFIDQGNRPLFEDMTDSDCRDNAPRTIFIISMYKDSQPRGM
AVTISVKCEKISTLSCENKIISFKEMNPPDNIKDTKSDIIFFQRSVPGHDNKMQFESSSYEG
YFLACEKERDLFKLILKKEDELGDRSIMFTVQNED
(SEQ ID NO: 35)
SEQ ID NO: 36
Decoy Resistant IL-18 polypeptide # 1
YFGKLESKLSVIRNLNDQVLFIDQGNRPLFEDMTDSDCRDNAPRTIFIISKYSDSLARGLA
VTISVKCEKISTLSCENKIISFKEMNPPDNIKDTKSDIIFFQRDVPGHSRKMQFESSSYEGY
FLACEKERDLFKLILKKEDELGDRSIMFTVQNED
(SEQ ID NO: 36)
SEQ ID NO: 37
Decoy Resistant IL-18 polypeptide # 2
YFGKLESKLSVIRNLNDQVLFIDQGNRPLFEDMTDSDSRDNAPRTIFIISKYSDSLARGLA
VTISVKSEKISTLSCENKIISFKEMNPPDNIKDTKSDIIFFQRDVPGHSRKMQFESSSYEGY
FLACEKERDLFKLILKKEDELGDRSIMFTVQNED
(SEQ ID NO: 37)
SEQ ID NO: 38
Decoy Resistant IL-18 polypeptide #3
YFGKLESKLSVIRNLNDQVLFIDQGNRPLFEDMTDSDSRDNAPRTIFIISKYSDSLARGLA
VTISVKGEKISTLSCENKIISFKEMNPPDNIKDTKSDIIFFQRDVPGHSRKMQFESSSYEGY
FLACEKERDLFKLILKKEDELGDRSIMFTVQNED
(SEQ ID NO: 38)
SEQ ID NO: 39
Decoy Resistant IL-18 polypeptide #4
YFGKLESKLSVIRNLNDQVLFIDQGNRPLFEDMTDSDSRDNAPRTIFIISKYSDSLARGLA
VTISVKAEKISTLSCENKIISFKEMNPPDNIKDTKSDIIFFQRDVPGHSRKMQFESSSYEGY
FLACEKERDLFKLILKKEDELGDRSIMFTVQNED
(SEQ ID NO: 39)
SEQ ID NO: 40
Decoy Resistant IL-18 polypeptide #5
YFGKLESKLSVIRNLNDQVLFIDQGNRPLFEDMTDSDSRDNAPRTIFIISKYSDSLARGLA
VTISVKVEKISTLSCENKIISFKEMNPPDNIKDTKSDIIFFQRDVPGHSRKMQFESSSYEGY
FLACEKERDLFKLILKKEDELGDRSIMFTVQNED
(SEQ ID NO: 40)
SEQ ID NO: 41
Decoy Resistant IL-18 polypeptide #6
YFGKLESKLSVIRNLNDQVLFIDQGNRPLFEDMTDSDSRDNAPRTIFIISKYSDSLARGLA
VTISVKDEKISTLSCENKIISFKEMNPPDNIKDTKSDIIFFQRDVPGHSRKMQFESSSYEGY
FLACEKERDLFKLILKKEDELGDRSIMFTVQNED
(SEQ ID NO: 41)
SEQ ID NO: 42
Decoy Resistant IL-18 polypeptide #7
YFGKLESKLSVIRNLNDQVLFIDQGNRPLFEDMTDSDSRDNAPRTIFIISKYSDSLARGLA
VTISVKEEKISTLSCENKIISFKEMNPPDNIKDTKSDIIFFQRDVPGHSRKMQFESSSYEGY
FLACEKERDLFKLILKKEDELGDRSIMFTVQNED
(SEQ ID NO: 42)
SEQ ID NO: 43
Decoy Resistant IL-18 polypeptide #8
YFGKLESKLSVIRNLNDQVLFIDQGNRPLFEDMTDSDSRDNAPRTIFIISKYSDSLARGLA
VTISVKNEKISTLSCENKIISFKEMNPPDNIKDTKSDIIFFQRDVPGHSRKMQFESSSYEGY
FLACEKERDLFKLILKKEDELGDRSIMFTVQNED
(SEQ ID NO: 43)

What is claimed is:

1. A method of treating a disease in a subject in need thereof, the method comprising: (a) administering successive doses of an anti-PD-1 antibody composition comprising pembrolizumab to the subject; and (b) administering successive doses of a decoy-resistant (DR) IL-18 composition comprising a polypeptide to the subject, wherein the polypeptide is a modified IL-18 polypeptide comprising an amino acid sequence set forth in any one of SEQ ID NOs: 36-43; thereby causing immunotherapy-induced regression of the disease in the subject.

2. The method of claim 1, further comprising administering a dose of a cytokine release syndrome (CRS) prophylactic agent to the subject.

3. The method of any one of the preceding claims, wherein the method comprises administering multiple doses of a CRS prophylactic agent to the subject comprising at least a dose of a NSAID and at least a dose of a histamine H1 antagonist; at least a dose of acetaminophen and at least a dose of a histamine H1 antagonist; at least a dose of a NSAID and at least a dose of acetaminophen; or at least a dose of a NSAID, at least a dose of a histamine H1 antagonist, and at least a dose of acetaminophen, optionally wherein the NSAID is selected from indomethacin and ibuprofen and the histamine H1 antagonist is diphenhydramine, optionally further comprising at least a dose of an H2 blocker, optionally wherein the H2 blocker is famotidine, optionally wherein the famotidine is oral famotidine administered at a 20 mg to 40 mg dose or intravenous famotidine administered at a 20 mg dose.

4. The method of claim 3, wherein:
(i) the NSAID is oral indomethacin administered at 50 mg to 100 mg per day, optionally at 75 mg per day, or oral ibuprofen administered at 600 mg to 2,400 mg per day, optionally 800 mg to 1,800 mg per day;
(ii) the histamine H1 antagonist is intravenous or oral diphenhydramine administered at a dose of 25 mg to 100 mg, optionally 50 mg;
(iii) the acetaminophen is oral acetaminophen administered at 350 mg to 4,000 mg per day, optionally 650 mg to 3,900 mg per day; or
(iv) any combination thereof.

5. The method of any one of the preceding claims, wherein each dose of the successive doses of the anti-PD-1 antibody composition is administered to the subject about every three weeks, wherein the anti-PD-1 antibody composition comprises 200 mg of pembrolizumab.

6. The method of any one of the preceding claims, wherein each dose of the successive doses of the DR IL-18 composition is administered to the subject weekly.

7. The method of any one of the preceding claims, wherein the polypeptide is a modified IL-18 polypeptide comprising, or consisting of, the amino acid sequence set forth in SEQ ID NO: 41.

8. The method of any one of the preceding claims, wherein a dose of the DR IL-18 composition comprises at least about 30 μg of the polypeptide per kg body weight of the subject.

9. The method of any one of the preceding claims, wherein the disease comprises cancer or a solid tumor.

10. The method of any one of the preceding claims, comprising administering one or more doses of a CRS prophylactic agent to the subject with or before each dose of the successive doses of the DR IL-18 composition, wherein the CRS prophylactic agent comprises at least one of a NSAID, a histamine H1 antagonist, acetaminophen, or a H2 blocker, and wherein:
(i) the polypeptide is a modified IL-18 polypeptide comprising or consisting of the amino acid sequence set forth in SEQ ID NO:41;
(ii) the dose of the DR IL-18 composition comprises at least about 30 μg of the polypeptide per kg body weight of the subject;
(iii) the dose of the anti-PD-1 antibody composition comprises at least about 200 mg of pembrolizumab; and
(iv) the disease comprises a PD-1 checkpoint inhibitor resistant solid tumor selected from the group consisting of: melanoma, Merkel cell carcinoma, RCC, urothelial, NSCLC, TNBC, SCCHN, MSI-H tumor, TMB-H or mismatch repair deficient tumor, gastric, cervical, endometrial, cutaneous squamous, small cell lung, esophageal, HCC, or any combination thereof, or platinum-resistant ovarian cancer or microsatellite stable colorectal cancer.

11. A method of treating a disease in a human patient comprising administering to the patient an anti-PD-1 antibody, or antigen binding fragment thereof, in combination with a decoy-resistant (DR) IL-18 composition comprising a polypeptide to the subject, wherein the polypeptide is a modified IL-18 polypeptide comprising an amino acid sequence set forth in any one of SEQ ID NOs: 36-43, wherein the anti-PD-1 antibody, or antigen binding fragment thereof, comprises a light chain complementarity determining regions (CDRs) comprising a sequence of amino acids as set forth in SEQ ID NOs: 1, 2 and 3 and heavy chain CDRs comprising a sequence of amino acids as set forth in SEQ ID NOs: 6, 7 and 8.

12. The method of claim 11, wherein the anti-PD-1 antibody, or antigen binding fragment thereof, is administered to the patient at a dose of about 200 mg once every three weeks.

13. The method of claim 11 or 12, wherein the anti-PD-1 antibody, or antigen binding fragment thereof, is pembrolizumab or a pembrolizumab variant.

14. The method of any one of claims 11-13, wherein the DR IL-18 composition is administered to the patient weekly, optionally wherein the polypeptide is a modified IL-18 polypeptide comprising, or consisting of, the amino acid sequence set forth in SEQ ID NO:41, optionally wherein the DR IL-18 composition is administered to the patient at a dose of at least about 30 μg of the polypeptide per kg body weight of the patient.

15. The method of any one of claims 11-14, wherein the disease comprises cancer or a solid tumor.

16. The method of claim 15, wherein the cancer or solid tumor is, or is associated with, melanoma, Merkel cell carcinoma, RCC, urothelial, NSCLC (with no epidermal growth factor receptor, TRK receptor, or anaplastic lymphoma kinase positive mutations/fusions), TNBC, SCCHN, MSI-H, TMB-H or mismatch repair deficient, gastric, cervical, endometrial, cutaneous squamous, small cell lung, esophageal, HCC, or any combination thereof, and wherein the cancer or solid tumor is resistant to PD-1 checkpoint inhibitors.

17. The method of claim 15, wherein the cancer is a hematological cancer, optionally a myeloma, a lymphoma, or a leukemia.

18. The method of any one of claims 11-17, further comprising administering a cytokine release syndrome (CRS) prophylactic agent to the patient, optionally wherein the CRS prophylactic agent comprises at least one of a nonsteroidal anti-inflammatory drug (NSAID), acetaminophen, histamine H1 antagonist, or H2 blocker.

19. The method of claim 18, wherein the method comprises administering multiple CRS prophylactic agents to the patient comprising at least a NSAID and at least a histamine H1 antagonist; at least acetaminophen and at least a histamine H1 antagonist; at least a NSAID and at least acetaminophen; or at least a NSAID, at least a histamine H1 antagonist, and at least acetaminophen, optionally wherein the NSAID is selected from indomethacin and ibuprofen and the histamine H1 antagonist is diphenhydramine, optionally further comprising at least an H2 blocker, optionally wherein the H2 blocker is famotidine.

20. The method of claim 19, wherein:
(i) the NSAID is oral indomethacin administered at 50 mg to 100 mg per day, optionally at 75 mg per day, or oral ibuprofen administered at 600 mg to 2,400 mg per day, optionally 800 mg to 1,800 mg per day;
(ii) the histamine H1 antagonist is intravenous or oral diphenhydramine administered in a dose of 25 mg to 100 mg, optionally 50 mg;
(iii) the acetaminophen is oral acetaminophen administered at 350 mg to 4,000 mg per day, optionally 650 mg to 3,900 mg per day; or
(iv) any combination thereof.

21. The method of any one of claims 11-20, comprising administering one or more CRS prophylactic agents to the patient with or before the DR IL-18 composition, wherein the one or more CRS prophylactic agents comprise a NSAID, a histamine H1 antagonist, acetaminophen, a H2 blocker, or a combination thereof, and wherein:
(i) the polypeptide is a modified IL-18 polypeptide comprising or consisting of the amino acid sequence set forth in SEQ ID NO:41;
(ii) the dose of the DR IL-18 composition comprises at least about 30 μg of the polypeptide per kg body weight of the patient;
(iii) the dose of the anti-PD-1 antibody composition comprises at least about 200 mg of pembrolizumab or a pembrolizumab variant; and
(iv) the disease comprises a PD-1 checkpoint inhibitor resistant solid tumor selected from the group consisting of: melanoma, Merkel cell carcinoma, RCC, urothelial, NSCLC, TNBC, SCCHN, MSI-H tumor, TMB-H or mismatch repair deficient tumor, gastric, cervical, endometrial, cutaneous squamous, small cell lung, esophageal, HCC, or any combination thereof; or platinum-resistant ovarian cancer or microsatellite stable colorectal cancer.


SEQUENCE LISTING (43 sequences)

SEQ ID NO: 1 - protein, 15 aa, synthetic construct
RASKGVSTSGYSYLH

SEQ ID NO: 2 - protein, 7 aa, synthetic construct
LASYLES

SEQ ID NO: 3 - protein, 9 aa, synthetic construct
QHSRDLPLT

SEQ ID NO: 4 - protein, 111 aa, synthetic construct [sequence omitted]

SEQ ID NO: 5 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
NYYMY

SEQ ID NO: 7 - protein, 17 aa, synthetic construct
GINPSNGGTNFNEKFKN

SEQ ID NO: 8 - protein, 11 aa, synthetic construct
RDYRFDMGFDY

SEQ ID NO: 9 - protein, 120 aa, synthetic construct [sequence omitted]

SEQ ID NO: 10 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11-20 - skipped (10 entries)

SEQ ID NO: 21 - protein, 15 aa, synthetic construct
RASKSVSTSGFSYLH

SEQ ID NO: 22 - protein, 7 aa, synthetic construct
LASNLES

SEQ ID NO: 23 - protein, 9 aa, synthetic construct
QHSWELPLT

SEQ ID NO: 24 - protein, 5 aa, synthetic construct
SYYLY

SEQ ID NO: 25 - protein, 17 aa, synthetic construct
GVNPSNGGTNFSEKFKS

SEQ ID NO: 26 - protein, 11 aa, synthetic construct
RDSNYDGGFDY

SEQ ID NO: 27 - protein, 120 aa, synthetic construct [sequence omitted]

SEQ ID NO: 28 - protein, 111 aa, synthetic construct [sequence omitted]

SEQ ID NO: 29 - protein, 109 aa, synthetic construct
EIVLTQSPLSLPVTPGEPASISCASKGVSTSGYSYLHWYLQKPGQSPQLLIYLASYLESGVPDRFSGSGSGTDFTLKISRVEAEDVGVYYCHSRDLPLTFGQGTKLEIK

SEQ ID NO: 30 - protein, 110 aa, synthetic construct
DIVMTQTPLSLPVTPGEPASISCASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 31 - protein, 442 aa, synthetic construct
QVQLVQSGVEVKKPGASVKVSCASGYTFTNYYMWVRQAPGQGLEWMGGINPSNGGTNFNEKFKNRVTLTTDSSTTTAYMELKSLQFDDTAVYYCARRDYRFDMFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMSRTPEVTCVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 218 aa, synthetic construct; identical to SEQ ID NO: 5

SEQ ID NO: 33 - protein, 215 aa, synthetic construct
EIVLTQSPLSLPVTPGEPASISCRASKGVSTSGYSYLHWYLQKPGQSPQLLIYLASYLESGVPDRFSGSGSGTDFTLKISRVEAEDVGVYYCHSRDLPLTFGQGTKLEIKRTVAAPSVFIFPPSDEQLKSGTASVVCLNNFYPREAKVQWKVDNALQSGNSQESVTEQDSKDSTYSLSSTLTLSKADYEKHKVYACETHQGLSSPVTKSFNRGEC

SEQ ID NO: 34 - protein, 218 aa, synthetic construct
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 36 - protein, 157 aa, synthetic construct
YFGKLESKLSVIRNLNDQVLFIDQGNRPLFEDMTDSDCRDNAPRTIFIISKYSDSLARGLAVTISVKCEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 37 - protein, 157 aa, synthetic construct
YFGKLESKLSVIRNLNDQVLFIDQGNRPLFEDMTDSDSRDNAPRTIFIISKYSDSLARGLAVTISVKSEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 38 - protein, 157 aa, synthetic construct
YFGKLESKLSVIRNLNDQVLFIDQGNRPLFEDMTDSDSRDNAPRTIFIISKYSDSLARGLAVTISVKGEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 39 - protein, 157 aa, synthetic construct
YFGKLESKLSVIRNLNDQVLFIDQGNRPLFEDMTDSDSRDNAPRTIFIISKYSDSLARGLAVTISVKAEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 40 - protein, 157 aa, synthetic construct
YFGKLESKLSVIRNLNDQVLFIDQGNRPLFEDMTDSDSRDNAPRTIFIISKYSDSLARGLAVTISVKVEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 41 - protein, 157 aa, synthetic construct
YFGKLESKLSVIRNLNDQVLFIDQGNRPLFEDMTDSDSRDNAPRTIFIISKYSDSLARGLAVTISVKDEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 42 - protein, 157 aa, synthetic construct
YFGKLESKLSVIRNLNDQVLFIDQGNRPLFEDMTDSDSRDNAPRTIFIISKYSDSLARGLAVTISVKEEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 43 - protein, 157 aa, synthetic construct
YFGKLESKLSVIRNLNDQVLFIDQGNRPLFEDMTDSDSRDNAPRTIFIISKYSDSLARGLAVTISVKNEKISTLSCENKIISFKEMNPPDNIKDTKSDIIFFQRDVPGHSRKMQFESSSYEGYFLACEKERDLFKLILKKEDELGDRSIMFTVQNED